SEQUENCE LISTING (50 sequences)

SEQ ID NO: 1 - DNA, 25 bp, Artificial sequence
  Single strand DNA oligonucleotide
caccgtagaatcccaggattcaggc

SEQ ID NO: 2 - DNA, 25 bp, Artificial sequence
  Single strand DNA oligonucleotide
aaacgcctgaatcctgggattctac

SEQ ID NO: 3 - DNA, 44 bp, Artificial sequence
  Single strand DNA oligonucleotide
ctcgaggtcgaccactattctgccatcctgcaggtcctacatcg

SEQ ID NO: 4 - DNA, 45 bp, Artificial sequence
  Single strand DNA oligonucleotide
ggtggcaagcttggcgggtggtaaagtggcaacggcgaatttggg

SEQ ID NO: 5 - DNA, 37 bp, Artificial sequence
  Single strand DNA oligonucleotide
cccgccaagcttgccaccatggtgagcaagggcgagg

SEQ ID NO: 6 - DNA, 46 bp, Artificial sequence
  Single strand DNA oligonucleotide
gattcaggatccagctcgagatctgagtccggacttgtacagctcg

SEQ ID NO: 7 - DNA, 46 bp, Artificial sequence
  Single strand DNA oligonucleotide
cgagctggatcctgaatcctgggattctagtatgcaataagagatg

SEQ ID NO: 8 - DNA, 46 bp, Artificial sequence
  Single strand DNA oligonucleotide
ggccgctctagagcagtgagccaagaccaggctactgcactccagc

SEQ ID NO: 9 - DNA, 24 bp, Artificial sequence
  Single strand DNA oligonucleotide
caccggacccttaatgatgcaggt

SEQ ID NO: 10 - DNA, 24 bp, Artificial sequence
  Single strand DNA oligonucleotide
aaacacctgcatcattaagggtcc

SEQ ID NO: 11 - DNA, 103 bp, Artificial sequence
  Single strand DNA oligonucleotide
tctgagcagagactcacccgtttataatagttctttatcttggttgccatgtcaacctgcatcattaagggtccattttcctcactatattctgcaagaataa

SEQ ID NO: 12 - DNA, 26 bp, Artificial sequence
  Single strand DNA oligonucleotide
gcagaacccatacatggatatggagg

SEQ ID NO: 13 - DNA, 26 bp, Artificial sequence
  Single strand DNA oligonucleotide
tatggtatatgttcacagattaccag

SEQ ID NO: 14 - DNA, 25 bp, Artificial sequence
  Single strand DNA oligonucleotide
caccgcttaatgatgcaggttgaca

SEQ ID NO: 15 - DNA, 25 bp, Artificial sequence
  Single strand DNA oligonucleotide
aaactgtcaacctgcatcattaagc

SEQ ID NO: 16 - DNA, 100 bp, Artificial sequence
  Single strand DNA oligonucleotide
ctgagcagagactcacccgtttataatagttctttatcttggttgccatgccaacctgcatcattaagggtccattttcctcactatattctgcaagaat

SEQ ID NO: 17 - DNA, 25 bp, Artificial sequence
  Single strand DNA oligonucleotide
caccgctgcacgccgtgggtcaggg

SEQ ID NO: 18 - DNA, 25 bp, Artificial sequence
  Single strand DNA oligonucleotide
aaacccctgacccacggcgtgcagc

SEQ ID NO: 19 - DNA, 99 bp, Artificial sequence
  Single strand DNA oligonucleotide
accggcaagctgcccgtgccctggcccaccctcgtgaccaccctgacatacggcgtgcagtgcttcagccgctaccccgaccacatgaagcagcacgac

SEQ ID NO: 20 - protein, 9 aa, Artificial sequence
  Meganuclease family structural motif sequence
LAGLIDADG

SEQ ID NO: 21 - protein, 6 aa, Artificial sequence
  Meganuclease family structural motif sequence
GIYYIG

SEQ ID NO: 22 - DNA, 8 bp, Artificial sequence
  PAM sequence of Neisseria meningitidis-derived Cas9
  misc_feature (1)..(4): n is a, c, g, or t
nnnngatt

SEQ ID NO: 23 - DNA, 6 bp, Artificial sequence
  PAM sequence of Streptococcus thermophilus- derived Cas9
  misc_feature (1)..(2): n is a, c, g, or t
nnagaa

SEQ ID NO: 24 - DNA, 6 bp, Artificial sequence
  PAM sequence of Treponema denticola-derived Cas9
  misc_feature (1)..(1): n is a, c, g, or t
naaaac

SEQ ID NO: 25 - protein, 626 aa, Artificial sequence
  AMINO ACID SEQ OF UL12
MESTGGPACPPGRTVTKRSWALAEDTPRGPDSPPKRPRPNSLPLTTTFRPLPPPPQTTSAVDPSSHSPVNPPRDQHATDTADEKPRAASPALSDASGPPTPDIPLSPGGTHARDPDADPDSPDLDSMWSASVIPNALPSHILAETFERHLRGLLRGVRAPLAIGPLWARLDYLCSLAVVLEEAGMVDRGLGRHLWRLTRRGPPAAADAVAPRPLMGFYEAATQNQADCQLWALLRRGLTTASTLRWGPQGPCFSPQWLKHNASLRPDVQSSAVMFGRVNEPTARSLLFRYCVGRADDGGEAGADTRRFIFHEPGDLAEENVHTCGVLMDGHTGMVGASLDILVCPRDIHGYLAPVPKTPLAFYEVKCRAKYAFDPMDPSDPTASAYEDLMAHRSPEAFRAFIRSIPKPSVRYFAPGRVPGPEEALVTQDQAWSEAHASGEKRRCSAADRALVELNSGVVSEVLLFGAPDLGRHTISPVSWSSGDLVRREPVFANPRHPNFKQILVQGYVLDSHFPDCPPHPHLVTFIGRHRTSAEEGVTFRLEDGAGALGAAGPSKASILPNQAVPIALIITPVRIDPEIYKAIQRSSRLAFDDTLAELWASRSPGPGPAAAETTSSSPTTGRSSR

SEQ ID NO: 26 - protein, 126 aa, Artificial sequence
  THE FIRST 126 AA OF UL12 - AMINO ACIDS SEQUENCE
MESTGGPACPPGRTVTKRSWALAEDTPRGPDSPPKRPRPNSLPLTTTFRPLPPPPQTTSAVDPSSHSPVNPPRDQHATDTADEKPRAASPALSDASGPPTPDIPLSPGGTHARDPDADPDSPDLDS

SEQ ID NO: 27 - protein, 1553 aa, Artificial sequence
  THE COMPLETE CAS9-UL12 FUSION PROTEIN U3- AMINO ACIDS SEQUENCE
MESTGGPACPPGRTVTKRSWALAEDTPRGPDSPPKRPRPNSLPLTTTFRPLPPPPQTTSAVDPSSHSPVNPPRDQHATDTADEKPRAASPALSDASGPPTPDIPLSPGGTHARDPDADPDSPDLDSGSVMDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKL

SEQ ID NO: 28 - protein, 1548 aa, Artificial sequence
  THE COMPLETE CAS9-UL12 FUSION PROTEIN U4- AMINO ACIDS SEQUENCE
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKMESTGGPACPPGRTVTKRSWALAEDTPRGPDSPPKRPRPNSLPLTTTFRPLPPPPQTTSAVDPSSHSPVNPPRDQHATDTADEKPRAASPALSDASGPPTPDIPLSPGGTHARDPDADPDSPDLDS

SEQ ID NO: 29 - protein, 1872 aa, Artificial sequence
  An exemplary sequence of TAF1
  misc_feature (695)..(695): Xaa can be any naturally occurring amino acid
MGPGCDLLLRTAATITAAAIMSDTDSDEDSAGGGPFSLAGFLFGNINGAGQLEGESVLDDECKKHLAGLGALGLGSLITELTANEELTGTDGALVNDEGWVRSTEDAVDYSDINEVAEDESRRYQQTMGSLQPLCHSDYDEDDYDADCEDIDCKLMPPPPPPPGPMKKDKDQDSITGEKVDFSSSSDSESEMGPQEATQAESEDGKLTLPLAGIMQHDATKLLPSVTELFPEFRPGKVLRFLRLFGPGKNVPSVWRSARRKRKKKHRELIQEEQIQEVECSVESEVSQKSLWNYDYAPPPPPEQCLSDDEITMMAPVESKFSQSTGDIDKVTDTKPRVAEWRYGPARLWYDMLGVPEDGSGFDYGFKLRKTEHEPVIKSRMIEEFRKLEENNGTDLLADENFLMVTQLHWEDDIIWDGEDVKHKGTKPQRASLAGWLPSSMTRNAMAYNVQQGFAATLDDDKPWYSIFPIDNEDLVYGRWEDNIIWDAQAMPRLLEPPVLTLDPNDENLILEIPDEKEEATSNSPSKESKKESSLKKSRILLGKTGVIKEEPQQNMSQPEVKDPWNLSNDEYYYPKQQGLRGTFGGNIIQHSIPAVELRQPFFPTHMGPIKLRQFHRPPLKKYSFGALSQPGPHSVQPLLKHIKKKAKMREQERQASGGGEMFFMRTPQDLTGKDGDLILAEYSEENGPLMMQVXMATKIKNYYKRKPGKDPGAPDCKYGETVYCHTSPFLGSLHPGQLLQAFENNLFRAPIYLHKMPETDFLIIRTRQGYYIRELVDIFVVGQQCPLFEVPGPNSKRANTHIRDFLQVFIYRLFWKSKDRPRRIRMEDIKKAFPSHSESSIRKRLKLCADFKRTGMDSNWWVLKSDFRLPTEEEIRAMVSPEQCCAYYSMIAAEQRLKDAGYGEKSFFAPEEENEEDFQMKIDDEVRTAPWNTTRAFIAAMKGKCLLEVTGVADPTGCGEGFSYVKIPNKPTQQKDDKEPQPVKKTVTGTDADLRRLSLKNAKQLLRKFGVPEEEIKKLSRWEVIDVVRTMSTEQARSGEGPMSKFARGSRFSVAEHQERYKEECQRIFDLQNKVLSSTEVLSTDTDSSSAEDSDFEEMGKNIENMLQNKKTSSQLSREREEQERKELQRMLLAAGSAASGNNHRDDDTASVTSLNSSATGRCLKIYRTFRDEEGKEYVRCETVRKPAVIDAYVRIRTTKDEEFIRKFALFDEQHREEMRKERRRIQEQLRRLKRNQEKEKLKGPPEKKPKKMKERPDLKLKCGACGAIGHMRTNKFCPLYYQTNAPPSNPVAMTEEQEEELEKTVIHNDNEELIKVEGTKIVLGKQLIESADEVRRKSLVLKFPKQQLPPKKKRRVGTTVHCDYLNRPHKSIHRRRTDPMVTLSSILESIINDMRDLPNTYPFHTPVNAKVVKDYYKIITRPMDLQTLRENVRKRLYPSREEFREHLELIVKNSATYNGPKHSLTQISQSMLDLCDEKLKEKEDKLARLEKAINPLLDDDDQVAFSFILDNIVTQKMMAVPDSWPFHHPVNKKFVPDYYKVIVNPMDLETIRKNISKHKYQSRESFLDDVNLILANSVKYNGPESQYTKTAQEIVNVCYQTLTEYDEHLTQLEKDICTAKEAALEEAELESLDPMTPGPYTPQPPDLYDTNTSLSMSRDASVFQDESNMSVLDIPSATPEKQVTQEGEDGDGDLADEEEGTVQQPQASVLYEDLLMSEGEDDEEDAGSDEEGDNPFSAIQLSESGSDSDVGSGGIRPKQPRMLQENTRMDMENEESMMSYEGDGGEASHGLEDSNISYGSYEEPDPKSNTQDTSFSSIGGYEVSEEEEDEEEEEQRSGPSVLSQVHLSEDEEDSEDFHSIAGDSDLDSDE

SEQ ID NO: 30 - DNA, 7599 bp, Artificial sequence
  An exemplary sequence of TAF1
atcactgctgccgccatcatgtcagacacggacagcgacgaagattccgctggaggcggcccattttctttagcgggtttccttttcggcaacatcaatggagccgggcagctggagggggaaagcgtcttggatgatgaatgtaagaagcacttggcaggcttgggggctttggggctgggcagcctgatcactgaactcacggcaaatgaagaattgaccgggactgacggtgccttggtaaatgatgaagggtgggttaggagtacagaagatgctgtggactattcagacatcaatgaggtggcagaagatgaaagccgaagataccagcagacgatggggagcttgcagcccctttgccactcagattatgatgaagatgactatgatgctgattgtgaagacattgattgcaagttgatgcctcctccacctccacccccgggaccaatgaagaaggataaggaccaggattctattactggtgtgtctgaaaatggagaaggcatcatcttgccctccatcattgccccttcctctttggcctcagagaaagtggacttcagtagttcctctgactcagaatctgagatgggacctcaggaagcaacacaggcagaatctgaagatggaaagctgacccttccattggctgggattatgcagcatgatgccaccaagctgttgccaagtgtcacagaactttttccagaatttcgacctggaaaggtgttacgttttctacgtctttttggaccagggaagaatgtcccatctgtttggcggagtgctcggagaaagaggaagaagaagcaccgtgagctgatacaggaagagcagatccaggaggtggagtgctcagtagaatcagaagtcagccagaagtctttgtggaactacgactacgctccaccaccacctccagagcagtgtctctctgatgatgaaatcacgatgatggctcctgtggagtccaaattttcccaatcaactggagatatagataaagtgacagataccaaaccaagagtggctgagtggcgttatgggcctgcccgactgtggtatgatatgctgggtgtccctgaagatggcagtgggtttgactatggcttcaaactgagaaagacagaacatgaacctgtgataaaatctagaatgatagaggaatttaggaaacttgaggaaaacaatggcactgatcttctggctgatgaaaacttcctgatggtgacacagctgcattgggaggatgatatcatctgggatggggaggatgtcaaacacaaagggacaaaacctcagcgtgcaagcctggcaggctggcttccttctagcatgactaggaatgcgatggcttacaatgttcagcaaggttttgcagccactcttgatgatgacaaaccttggtactccatttttcccattgacaatgaggatctggtatatggacgctgggaggacaatatcatttgggatgctcaggccatgccccggctgttggaacctcctgttttgacacttgatcccaatgatgagaacctcattttggaaattcctgatgagaaggaagaggccacctctaactccccctccaaggagagtaagaaggaatcatctctgaagaagagtcgaattctcttagggaaaacaggagtcatcaaggaggaaccacagcagaacatgtctcagccagaagtgaaagatccatggaatctctccaatgatgagtattattatcccaagcaacagggtcttcgaggcacctttggagggaatattatccagcattcaattcctgctgtggaattacggcagcccttctttcccacccacatggggcccatcaaactccggcagttccatcgcccacctctgaaaaagtactcatttggtgcactttctcagccaggtccccactcagtccaacctttgctaaagcacatcaaaaaaaaggccaagatgagagaacaagagaggcaagcttcaggtggtggagagatgttttttatgcgcacacctcaggacctcacaggcaaagatggtgatcttattcttgcagaatatagtgaggaaaatggacccttaatgatgcaggttggcatggcaaccaagataaagaactattataaacggaaacctggaaaagatcctggagcaccagattgtaaatatggggaaactgtttactgccatacatctcctttcctgggttctctccatcctggccaattgctgcaagcatttgagaacaacctttttcgtgctccaatttatcttcataagatgccagaaactgatttcttgatcattcggacaagacagggttactatattcgggaattagtggatatttttgtggttggccagcagtgtcccttgtttgaagttcctgggcctaactccaaaagggccaatacgcatattcgagactttctacaggtttttatttaccgccttttctggaaaagtaaagatcggccacggaggatacgaatggaagatataaaaaaagcctttccttcccattcagaaagcagcatccggaagaggctaaagctctgcgctgacttcaaacgcacagggatggactcaaactggtgggtgcttaagtctgattttcgtttaccaacggaagaagagatcagagctatggtgtcaccagagcagtgctgtgcttattatagcatgatagctgcagagcaacgactgaaggatgctggctatggtgagaaatccttttttgctccagaagaagaaaatgaggaagatttccagatgaagattgatgatgaagttcgcactgccccttggaacaccacaagggccttcattgctgccatgaagggcaagtgtctgctagaggtgactggggtggcagatcccacggggtgtggtgaaggattctcctatgtgaagattccaaacaaaccaacacagcagaaggatgataaagaaccgcagccagtgaagaagacagtgacaggaacagatgcagaccttcgtcgcctttccctgaaaaatgccaagcaacttctacgtaaatttggtgtgcctgaggaagagattaaaaagttgtcccgctgggaagtgattgatgtggtgcgcacaatgtcaacagaacaggctcgttctggagaggggcccatgagtaaatttgcccgtggatcaaggttttctgtggctgagcatcaagagcgttacaaagaggaatgtcagcgcatctttgacctacagaacaaggttctgtcatcaactgaagtcttatcaactgacacagacagcagctcagctgaagatagtgactttgaagaaatgggaaagaacattgagaacatgttgcagaacaagaaaaccagctctcagctttcacgtgaacgggaggaacaggagcggaaggaactacagcgaatgctactggcagcaggctcagcagcatccggaaacaatcacagagatgatgacacagcttccgtgactagccttaactcttctgccactggacgctgtctcaagatttatcgcacgtttcgagatgaagaggggaaagagtatgttcgctgtgagacagtccgaaaaccagctgtcattgatgcctatgtgcgcatacggactacaaaagatgaggaattcattcgaaaatttgccctttttgatgaacaacatcgggaagagatgcgaaaagaacggcggaggattcaagagcaactgaggcggcttaagaggaaccaggaaaaggagaagcttaagggtcctcctgagaagaagcccaagaaaatgaaggagcgtcctgacctaaaactgaaatgtggggcatgtggtgccattggacacatgaggactaacaaattctgccccctctattatcaaacaaatgcgccaccttccaaccctgttgccatgacagaagaacaggaggaggagttggaaaagacagtcattcataatgataatgaagaacttatcaaggttgaagggaccaaaattgtcttggggaaacagctaattgagagtgcggatgaggttcgcagaaaatctctggttctcaagtttcctaaacagcagcttcctccaaagaagaaacggcgagttggaaccactgttcactgtgactatttgaatagacctcataagtccatccaccggcgccgcacagaccctatggtgacgctgtcgtccatcttggagtctatcatcaatgacatgagagatcttccaaatacataccctttccacactccagtcaatgcaaaggttgtaaaggactactacaaaatcatcactcggccaatggacctacaaacactccgcgaaaacgtgcgtaaacgcctctacccatctcgggaagagttcagagagcatctggagctaattgtgaaaaatagtgcaacctacaatgggccaaaacactcattgactcagatctctcaatccatgctggatctctgtgatgaaaaactcaaagagaaagaagacaaattagctcgcttagagaaagctatcaaccccttgctggatgatgatgaccaagtggcgttttctttcattctggacaacattgtcacccagaaaatgatggcagttccagattcttggccatttcatcacccagttaataagaaatttgttccagattattacaaagtgattgtcaatccaatggatttagagaccatacgtaagaacatctccaagcacaagtatcagagtcgggagagctttctggatgatgtaaaccttattctggccaacagtgttaagtataatggacctgagagtcagtatactaagactgcccaggagattgtgaacgtctgttaccagacattgactgagtatgatgaacatttgactcaacttgagaaggatatttgtactgctaaagaagcagctttggaggaagcagaattagaaagcctggacccaatgaccccagggccctacacgcctcagcctcctgatttgtatgataccaacacatccctcagtatgtctcgagatgcctctgtatttcaagatgagagcaatatgtctgtcttggatattcccagtgccactccagaaaagcaggtaacacaggaaggtgaagatggagatggtgatcttgcagatgaagaggaaggaactgtacaacagcctcaagccagtgtcctgtatgaggatttgcttatgtctgaaggagaagatgatgaggaagatgctgggagtgatgaagaaggagacaatcctttctctgctatccagctgagtgaaagtggaagtgactctgatgtgggatctggtggaataagacccaaacaaccccgcatgcttcaggagaacacaaggatggacatggaaaatgaagaaagcatgatgtcctatgagggagacggtggggaggcttcccatggtttggaggatagcaacatcagttatgggagctatgaggagcctgatcccaagtcgaacacccaagacacaagcttcagcagcatcggtgggtatgaggtatcagaggaggaagaagatgaggaggaggaagagcagcgctctgggccgagcgtactaagccaggtccacctgtcagaggacgaggaggacagtgaggatttccactccattgctggggacagtgacttggactctgatgaatgaggcttcctttgggcctccttggtcagccttccctgttctccagcctaggtggttcacctttccccaatttgttcatatttgtacagtatctgatcctgaaatcatgaaattaactaacaccttagcctttttaaaagtagtaagtaaatgataataaatcacctctcctaatcttcctggggcaatgtcaccctttgatttaaaacaaagcaaccccctttcccctaccactacggaaaagagcaagctcatttttccgtgtcctcctttatttaactccatttattgcttttggtataatttttccctggggaaggaggggaaattatgaaagaactagtaactttatgtcctcttgatgtattaggaaatttccggccaggcgtggtggctcacacctgtaatctcagcactctgggaggccgaggcgggcagatcacctgaggtcagaagttcgagaccagcttggccaacatggcgaaaccgcatctctactaaaaatacaaaaattagccaggtgtggtggcgtatgcctgttaatcctagctactcgggaggctgaggcaggagaattacttgaacccgggaggcagaggttgcagtgagtggaggtcacgccactgcactccagcctgggcgaaagagtgagattcagtctcaaaaaaaaaaaaatttccaagcatggtatcatctcacttttctaatttacaggctggagcagatgagagccctcctgctgggacagagaattgggttctagtggactctgtgctacacttaaacctgtgagacaaaccgcccattattttattatttaattatgcaatgcctagttcctaaatggattggaggcaaattaccgtaaattttgaaacagcctatatgtcagaaatgataatgttgccacctaaatgttttctgtcccccccaccctccccaggggaaatggtaggaaaatggtaagtttcttagggcaaagactgtgtcttctgtttcttttcatgcttaggatatggttctgtgcatagtaggtactcagtaaatgttcctagaatcataaaatcctcaacagatatgttactgagcatctgcttttcatgataagcactctatcagatccttgggatgcaaaggtaaataagacaaatcccttttacccaaagagctcaccatcaagttgggggagggaaagtggaattcaaaacatgttaataaatcatcatagtactgtgagataagtgcaattaagaagctagttataaagtataggggaaatagaggagtaatcatgtctgaaaagtcaggaaagtcttcctagaggtaatttttaagctgattgttttagaattagtagaagcttgccagatggaaaagtccaggcaaagtgtaacatgaatgggaaaggccacagtctagaaatggcagagtgtgttcctagtttgtttgtttgtttgtttgtacctgccttgttccaggaaggatttaatgtggtttatattccagtcctttaatgctggaagggctgagatgagactgaaagatgggcaggaagtatatcatcacaagctttgtgtttgatgttaatgtgtatgatttttatattatgggaaataagctcttagaggagtgatataatcaggtttgtgttttagaaatctgtgtaatgaatgaatgaagaaagaaattgaagaatcatgtaacatatgtgatcgcatttttgtaaaagaaccatgtgtgtttatatgtgtttatatatatacttgtgtatgcaaaggtaaaagtctgaaaggatatatgctaactgttcacaatgataaccccccaggaatgggattggaggggagggggcttctgtgtttgttatgtatgctgggtgggatattgtgcttttatttctatattgtttgaatttttttacagtatgtattatttttgtaataaaaattttaaaaaattcca

SEQ ID NO: 31 - protein, 106 aa, Artificial sequence
  Insertion of a P54Q mutation on RPL36A  AA
  misc_feature (54)..(54): Xaa can be any naturally occurring amino acid
MVNVPKTRRTFCKKCGKHQPHKVTQYKKGKDSLYAQGKRRYDRKQSGYGGQTKXIFRKKAKTTKKIVLRLECVEPNCRSKRMLAIKRCKHFELGGDKKRKGQVIQF

SEQ ID NO: 32 - DNA, 761 bp, Artificial sequence
  protein a positive selectable marker which allows the growth of cells in the presence of cycloheximide nucleic acid sequence
ctttctttccgcgccgatagcgctcacgcaagcatggttaacgtccctaaaacccgccggactttctgtaagaagtgtggcaagcaccaaccccataaagtgacacagtacaagaagggcaaggattctctgtacgcccagggaaagcggcgttatgacaggaagcagagtggctatggtgggcaaactaagccgattttccggaaaaaggctaaaactacaaagaagattgtgctaaggcttgagtgcgttgagcccaactgcagatctaagagaatgctggctattaaaagatgcaagcattttgaactgggaggagataagaagagaaagggccaagtgatccagttctaagtgtcatcttttattatgaagacaataaaatcttgagtttatgttcacttcatttgtttgctgttcatcttttgggagggaataagctagagccatcaatacaattccgcttgtggggaaatttatgcctcttactggtactacttgttttgcattgaagctgactggttgagttcacatcatatgttgcaattttctaatttggcacttcaatcactaggggccttatgaggcagtttgtcattatgcaatggttattggttatcatgtgagtagacacatttcaggctaatagggagaagtcagtaacacattcatagtgaatatgagatgtctttgctaagagttaagtgtcagatctttgttataacagttaatttaataaagaattttggcattgttcttca

SEQ ID NO: 33 - protein, 187 aa, Artificial sequence
  encoded protein a positive selectable marker which allows the growth of cells in the presence of methotrexate.
  misc_feature (23)..(23): Xaa can be any naturally occurring amino acid
MVGSLNCIVAVSQNMGIGKNGDXPWPPLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 34 - protein, 389 aa, Artificial sequence
  encoded protein a positive selectable marker which allows the growth of cells in the presence of hydroxyurea
  misc_feature (128)..(128): Xaa can be any naturally occurring amino acid
MLSLRVPLAPITDPQQLQLSPLKGLSLVDKENTPPALSGTRVLASKTARRIFQEPTEPKTKAAAPGVEDEPLLRENPRRFVIFPIEYHDIWQMYKKAEASFWTAEEVDLSKDIQHWESLKPEERYFIXHVLAFFAASDGIVNENLVERFSQEVQITEARCFYGFQIAMENIHSEMYSLLIDTYIKDPKEREFLFNAIETMPCVKKKADWALRWIGDKEATYGERVVAFAAVEGIFFSGSFASIFWLKKRGLMPGLTFSNELISRDEGLHCDFACLMFKHLVHKPSEERVREIIINAVRIEQEFLTEALPVKLIGMNCTLMKQYIEFVADRLMLELGFSKVFRVENPFDFMENISLEGKTNFFEKRVGEYQRMGVMSSPTENSFTLDADF

SEQ ID NO: 35 - protein, 1970 aa, Artificial sequence
  POLR2A (largest subunit of RNA polymerase II)
MHGGGPPSGDSACPLRTIKRVQFGVLSPDELKRMSVTEGGIKYPETTEGGRPKLGGLMDPRQGVIERTGRCQTCAGNMTECPGHFGHIELAKPVFHVGFLVKTMKVLRCVCFFCSKLLVDSNNPKIKDILAKSKGQPKKRLTHVYDLCKGKNICEGGEEMDNKFGVEQPEGDEDLTKEKGHGGCGRYQPRIRRSGLELYAEWKHVNEDSQEKKILLSPERVHEIFKRISDEECFVLGMEPRYARPEWMIVTVLPVPPLSVRPAVVMQGSARNQDDLTHKLADIVKINNQLRRNEQNGAAAHVIAEDVKLLQFHVATMVDNELPGLPRAMQKSGRPLKSLKQRLKGKEGRVRGNLMGKRVDFSARTVITPDPNLSIDQVGVPRSIAANMTFAEIVTPFNIDRLQELVRRGNSQYPGAKYIIRDNGDRIDLRFHPKPSDLHLQTGYKVERHMCDGDIVIFNRQPTLHKMSMMGHRVRILPWSTFRLNLSVTTPYNADFDGDEMNLHLPQSLETRAEIQELAMVPRMIVTPQSNRPVMGIVQDTLTAVRKFTKRDVFLERGEVMNLLMFLSTWDGKVPQPAILKPRPLWTGKQIFSLIIPGHINCIRTHSTHPDDEDSGPYKHISPGDTKVVVENGELIMGILCKKSLGTSAGSLVHISYLEMGHDITRLFYSNIQTVINNWLLIEGHTIGIGDSIADSKTYQDIQNTIKKAKQDVIEVIEKAHNNELEPTPGNTLRQTFENQVNRILNDARDKTGSSAQKSLSEYNNFKSMVVSGAKGSKINISQVIAVVGQQNVEGKRIPFGFKHRTLPHFIKDDYGPESRGFVENSYLAGLTPTEFFFHAMGGREGLIDTAVKTAETGYIQRRLIKSMESVMVKYDATVRNSINQVVQLRYGEDGLAGESVEFQNLATLKPSNKAFEKKFRFDYTNERALRRTLQEDLVKDVLSNAHIQNELEREFERMREDREVLRVIFPTGDSKVVLPCNLLRMIWNAQKIFHINPRLPSDLHPIKVVEGVKELSKKLVIVNGDDPLSRQAQENATLLFNIHLRSTLCSRRMAEEFRLSGEAFDWLLGEIESKFNQAIAHPGEMVGALAAQSLGEPATQMTLNTFHYAGVSAKNVTLGVPRLKELINISKKPKTPSLTVFLLGQSARDAERAKDILCRLEHTTLRKVTANTAIYYDPNPQSTVVAEDQEWVNVYYEMPDFDVARISPWLLRVELDRKHMTDRKLTMEQIAEKINAGFGDDLNCIFNDDNAEKLVLRIRIMNSDENKMQEEEEVVDKMDDDVFLRCIESNMLTDMTLQGIEQISKVYMHLPQTDNKKKIIITEDGEFKALQEWILETDGVSLMRVLSEKDVDPVRTTSNDIVEIFTVLGIEAVRKALERELYHVISFDGSYVNYRHLALLCDTMTCRGHLMAITRHGVNRQDTGPLMKCSFEETVDVLMEAAAHGESDPMKGVSENIMLGQLAPAGTGCFDLLLDAEKCKYGMEIPTNIPGLGAAGPTGMFFGSAPSPMGGISPAMTPWNQGATPAYGAWSPSVGSGMTPGAAGFSPSAASDASGFSPGYSPAWSPTPGSPGSPGPSSPYIPSPGGAMSPSYSPTSPAYEPRSPGGYTPQSPSYSPTSPSYSPTSPSYSPTSPNYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPSYSPTSPNYSPTSPNYTPTSPSYSPTSPSYSPTSPNYTPTSPNYSPTSPSYSPTSPSYSPTSPSYSPSSPRYTPQSPTYTPSSPSYSPSSPSYSPASPKYTPTSPSYSPSSPEYTPTSPKYSPTSPKYSPTSPKYSPTSPTYSPTTPKYSPTSPTYSPTSPVYTPTSPKYSPTSPTYSPTSPKYSPTSPTYSPTSPKGSTYSPTSPGYSPTSPTYSLTSPAISPDDSDEEN

SEQ ID NO: 36 - DNA, 30 bp, Artificial sequence
  Guide sequence
ggacccttaatgatgcaggttggcatggca

SEQ ID NO: 37 - DNA, 30 bp, Artificial sequence
  Guide sequence
ggacccttaatgatgcaggttgacatggca

SEQ ID NO: 38 - DNA, 51 bp, Artificial sequence
  partial nucleic acid sequence of PSMB6
tcgccgttgccactttaccacccgcctgaatcctgggattctagtatgcaa

SEQ ID NO: 39 - DNA, 33 bp, Artificial sequence
  Partial nucleic acid sequence of human RPL36A
actaagccgattttccggaaaaaggtgagtggt

SEQ ID NO: 40 - DNA, 31 bp, Artificial sequence
  An exemplary DHFR mutation in human
ggagacctaccctggcctccgctcaggtatc

SEQ ID NO: 41 - DNA, 31 bp, Artificial sequence
  An exemplary DHFR mutation in human
ggagacctaccctggcctccgctcaggtatt

SEQ ID NO: 42 - DNA, 33 bp, Artificial sequence
  Targeted locus of human R2
tttatatcccatgttctggctttctttgcagca

SEQ ID NO: 43 - DNA, 24 bp, Artificial sequence
  an example for edited nucleic acid sequence resistant to SiRNA
cttcttctacttctactactactt

SEQ ID NO: 44 - DNA, 24 bp, Artificial sequence
  an example for WT nucleic acid sequence susceptible to SiRNA
cttctactgctcctcctactactt

SEQ ID NO: 45 - DNA, 119 bp, Artificial sequence
  Wild-type PSMA1 3′ UTR
tcttccctttcccaggatctcacttgcttatctgaagaagattgtccaggctcatattgggaatgcttatgaggaaattcatgccgagacctgctattcaatgcatgtatcgttgcctc

SEQ ID NO: 46 - DNA, 119 bp, Artificial sequence
  donor ssODN
tcttccctttcccaggatctcacttgcttatctgaagaagattgtccaggctgatagtctgaattcatatgaggaaattcatgccgagacctgctattcaatgcatgtatcgttgcctc

SEQ ID NO: 47 - DNA, 25 bp, Artificial sequence
  humTAF1_tsmut_g2_forward
caccgcttaatgatgcaggttgaca

SEQ ID NO: 48 - DNA, 23 bp, Artificial sequence
  humTAF1_tsmut_g2_reverse
cttaatgatgcaggttgacatgg

SEQ ID NO: 49 - DNA, 100 bp, Artificial sequence; identical to SEQ ID NO: 16
  ssODN_humTAFwt  (-strand)

SEQ ID NO: 50 - DNA, 19 bp, Artificial sequence
  partial sequence of  PSMD1
taagcattcccaatatgag

(19) United States
(12) Patent Application Publication
(10) Pub. No.: US 2023/0313173 A1
(43) Pub. Date: Oct. 5, 2023

(54) SYSTEMS AND METHODS FOR IDENTIFYING CELLS THAT HAVE UNDERGONE GENOME EDITING

(71) Applicant: Yeda Research and Development Co. Ltd., Rehovot (IL)

(72) Inventors: Yosef SHAUL, Rehovot (IL); Nina REUVEN, Rehovot (IL)

(73) Assignee: Yeda Research and Development Co. Ltd., Rehovot (IL)

(21) Appl. No.: 17/787,977

(22) PCT Filed: Dec. 22, 2020
(86) PCT No.: PCT/IL2020/051322

§ 371 Date: Jun. 22, 2022

(30) Foreign Application Priority Data
Dec. 22, 2019 (IL) 271656

(51) Int. Cl. C12N 15/10; C12N 9/22; C12N 15/11; C12N 5/071; C12N 15/113

(52) U.S. Cl. CPC C12N 15/102; C12N 9/22; C12N 15/11; C12N 5/0686; C12N 15/113; C12N 2310/20; C12N 2510/00; C12N 2310/14

(57) ABSTRACT

Kits and methods for selecting a cell harboring a genome-editing event at a target sequence of interest are disclosed. One such kit comprises:
(i) a first DNA editing agent for specifically introducing a mutation into a first gene so as to generate a first selection marker which imparts resistance to a selection agent, wherein the target sequence of interest the said first gene are distinct; and
(ii) the selection agent; and/or
(iii) a second DNA editing agent for specifically introducing a mutation into the first gene, wherein the mutation disrupts selection marker activity of the first selection marker.

### RELATED APPLICATIONS

This application claims the benefit of priority of Israeli Patent Application No. 271656 filed 22 Dec., 2019, the contents of which are incorporated herein by reference in their entirety.

### SEQUENCE LISTING STATEMENT

The ASCII file, entitled 85175 Sequence Listing.txt, created on 21 Dec. 2020, comprising 94,705 bytes, submitted concurrently with the filing of this application is incorporated herein by reference.

### FIELD AND BACKGROUND OF THE INVENTION

The present invention, in some embodiments thereof, relates to a method of selecting cells which have undergone a genome editing event without leaving a selection marker footprint in the cell.

CRISPR (clustered regularly interspaced short palindromic repeat) and Cas (CRISPR-associated) proteins are part of the RNA-based adaptive immune system in bacteria and archaea. Cas9 is a DNA endonuclease, which is targeted to a specific target site by a short RNA guide sequence (sgRNA) complementary to the target sequence. The resulting double-strand break (DSB) is usually repaired by the endogenous cell repair machinery, either by non-homologous end joining (NHEJ) or homology directed repair (HDR), with NHEJ being the predominant repair pathway. NHEJ is highly efficient, but error prone, and produces small insertions or deletions (indels), generally resulting in frame shift mutations, thus generating a gene knockout. Homology-directed repair can occur if a donor template DNA with homology to the sequences flanking the DSB is provided, to produce edited sites with specific, targeted modifications (gene knock-in).

Nevertheless, introducing directed templated changes by homology-directed repair (HDR) requires the cellular DNA repair machinery, such as the MRN complex (Mre11/Rad50/Nbs1). HDR normally occurs only during the S/G2/M portion of the cell cycle, and thus in most cases, only a minority of the cells are competent to make templated genomic changes. Therefore, a way to select for these cells is needed.

Background art includes US Patent Application No. 20190225992; Serif et al., Nature Communications, 2018, 9:3924, DOI: 10.1038/s41467-018-06378-9; and Tapparia et al Nature Scientific Reports, (2019) 9:8217 | https://doi.org/10.1038/s41598-019-44710-5.

### SUMMARY OF THE INVENTION

According to an aspect of some embodiments of the present invention there is provided a kit for selecting a cell harboring a genome-editing event at a target sequence of interest, comprising:
(i) a first DNA editing agent for specifically introducing a mutation into a first gene so as to generate a first selection marker which imparts resistance to a selection agent, wherein the target sequence of interest and the first gene are distinct; and
(ii) the selection agent; and/or
(iii) a second DNA editing agent for specifically introducing a mutation into the first gene, wherein the mutation disrupts selection marker activity of the first selection marker.

According to an aspect of some embodiments of the present invention there is provided a kit for selecting a cell harboring a genome-editing event at a target sequence of interest, wherein a first gene of a genome of the transformed cell is mutated so as to render the first gene a first selection marker, wherein the kit comprises:
(i) a first DNA editing agent for specifically introducing a mutation into the first gene, wherein the mutation disrupts selection marker activity of the first selection marker, wherein the target sequence of interest and the first gene are distinct;
(ii) a second DNA editing agent for specifically introducing a mutation into the first gene so as to generate the first selection marker;
(iii) a third DNA editing agent for specifically introducing a mutation into a second gene of a genome of the transformed cell so as to render the second gene a second selection marker; and/or
(iv) a fourth DNA editing agent for specifically introducing a mutation into the second gene which disrupts marker activity of the second selection marker.

According to an aspect of some embodiments of the present invention there is provided a method of selecting a cell which harbors a genome-editing event at a target sequence of interest comprising:
(a) providing cells having a genome, the cells comprising a mutation on a first gene of the genome, the mutation rendering the first gene a selection marker;
(b) co-transfecting the cells with:
(i) a first DNA editing agent for specifically disrupting selection marker activity of the selection marker; and
(ii) a second DNA editing agent for specifically editing the genome at the target sequence of interest; and
(c) culturing the cells under conditions that enrich for cells that do not comprise the selection marker, thereby selecting a cell which harbors the genome-editing event.

According to an aspect of some embodiments of the present invention there is provided a method of selecting a cell which harbors a genome-editing event at a target sequence of interest comprising:
(a) co-transfecting cells having a genome with:
(i) a first DNA editing agent for specifically introducing a mutation on a first gene of the genome, the mutation rendering the first gene a selection marker which imparts resistance to an RNA silencing agent; and
(ii) a second DNA editing agent for specifically editing the genome at the target sequence of interest; and
(c) culturing the cells under conditions that enrich for cells that comprise the selection marker, thereby selecting a cell which harbors the genome-editing event.

According to an aspect of some embodiments of the present invention there is provided a method of selecting a cell which harbors a genome-editing event at a target sequence of interest comprising:
(a) co-transfecting cells having a genome with:
(i) a first DNA editing agent for introducing a first mutation into a first gene of a genome of the cells, the first mutation renders the first gene a first selection marker having a selection marker activity which imparts susceptibility of the cells to a condition;
(ii) a second DNA editing agent for introducing a second mutation into a second gene of a genome of the cells, the second mutation renders the second gene a second selection marker having a selection marker activity which imparts resistance of the cells to an agent; and
(iii) a third editing agent for editing the genome at the target sequence of interest;
(b) culturing the cells in the presence of the agent so as to enrich for cells that comprise the second selection marker;
(c) co-transfecting the cells that comprise the second selection marker with:
(i) a fourth DNA editing agent for disrupting the selection marker activity of the first selection marker;
(ii) a fifth DNA editing agent for disrupting the selection marker activity of the second selection marker; and
(d) culturing the cells under conditions that enrich for cells that do not comprise the first selection marker, thereby selecting a cell harboring the genome-editing event.

According to embodiments of the invention, the kit comprises:
(i) a first DNA editing agent for specifically introducing a mutation into a first gene, the first gene being an essential gene, so as to generate a first selection marker which imparts resistance to a selection agent, wherein the target sequence of interest and the first gene are distinct; and
(ii) the selection agent.

According to embodiments of the invention, the kit further comprises:
a selection agent, wherein the first selection marker or the second selection marker imparts resistance to the selection agent.

According to embodiments of the invention, the selection agent is an RNA silencing agent.

According to embodiments of the invention, the RNA silencing agent is an siRNA.

According to embodiments of the invention, the first gene is a housekeeping gene or an essential gene.

According to embodiments of the invention, the kit further comprises:
(iii) a third DNA editing agent for specifically introducing a mutation into a second gene of a genome of the transformed cell so as to render the second gene a second selection marker; and/or
(iv) a fourth DNA editing agent for specifically introducing a mutation into the second gene which disrupts marker activity of the second selection marker.

According to embodiments of the invention, the first selection marker imparts sensitivity to a condition or resistance to an agent.

According to embodiments of the invention, the first selection marker imparts sensitivity to a condition and the second selection marker imparts resistance to an agent or vice versa.

According to embodiments of the invention, the condition is a temperature.

According to embodiments of the invention, the first gene is selected from the group consisting of Transcription initiation factor TFIID subunit 1 (TAF1), E1 ubiquitin-activating enzyme, Ribosomal Protein L36a (RPL36A), dihydrofolate reductase (DHFR), RNA polymerase, ribonucleotide reductase (RNR), DNA polymerase and a proteasome subunit.

According to embodiments of the invention, the agent is selected from the group consisting of cycloheximide, methotrexate, hydroxyurea, Bortezomib, Carfilzomib, Ixazomib, Marizomib, Oprozomib, Delanzomib and alpha-Amanitin.

According to embodiments of the invention, the first gene is an essential gene and the agent is an RNA silencing agent directed towards the essential gene.

According to embodiments of the invention, the first DNA editing agent comprises a nuclease selected from the group consisting of a meganuclease (MN), a zinc finger nuclease (ZFN), a transcription activator-like effector nuclease (TALEN) and a clustered regularly interspaced short palindromic repeat (CRISPR)-associated nuclease (Cas9).

According to embodiments of the invention, the second DNA editing agent comprises a nuclease selected from the group consisting of a meganuclease (MN), a zinc finger nuclease (ZFN), a transcription activator-like effector nuclease (TALEN) and a clustered regularly interspaced short palindromic repeat (CRISPR)-associated nuclease (Cas9).

According to embodiments of the invention, the first and/or the second DNA editing agent comprise Cas9 and a guide RNA (gRNA).

According to embodiments of the invention, the first DNA editing agent further comprises a first DNA donor template comprising a nucleic acid sequence which encodes a wild-type sequence of the first gene.

According to embodiments of the invention, the second DNA editing agent further comprises a second DNA donor template comprising a nucleic acid sequence which encodes a mutated sequence of the first gene.

According to embodiments of the invention, the first DNA editing agent further comprises a first DNA donor template comprising a nucleic acid sequence which encodes a mutated sequence of the first gene and wherein the second DNA editing agent further comprises a second DNA donor template comprising a nucleic acid sequence which encodes a wild-type sequence of the first gene.

According to embodiments of the invention, the mutated sequence comprises a point mutation.

According to embodiments of the invention, the mutation is a point mutation.

According to embodiments of the invention, the first and/or the second DNA editing agent comprises a nuclease selected from the group consisting of a meganuclease (MN), a zinc finger nuclease (ZFN), a transcription activator-like effector nuclease (TALEN) and Cas9.

According to embodiments of the invention, the first and the second DNA editing agent comprise a gRNA and Cas9.

According to embodiments of the invention, the selection marker imparts susceptibility to a condition.

According to embodiments of the invention, the condition is a temperature.

According to embodiments of the invention, the selection marker imparts susceptibility to an agent.

According to embodiments of the invention, the first gene is an essential gene.

According to embodiments of the invention, the RNA silencing agent is siRNA.

According to embodiments of the invention, the cells comprise eukaryotic cells.

According to embodiments of the invention, the eukaryotic cells comprise human cells.

According to embodiments of the invention, the cells comprise stem cells.

According to embodiments of the invention, the cells comprise immune cells.

According to embodiments of the invention, the cells comprise diseased cells.

According to embodiments of the invention, the condition is a temperature.

According to embodiments of the invention, the agent is selected from the group consisting of cycloheximide, methotrexate, hydroxyurea, Bortezomib, Carfilzomib, Ixazomib, Marizomib, Oprozomib, Delanzomib and alpha-Amanitin.

According to embodiments of the invention, the genome editing event is mediated via homology-directed repair (HDR).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D. Schematic presentation of the co-editing principle. A. HDR editing of a selectable endogenous gene increases the probability of a second editing event in the cell. B. The experimental steps of co-editing in cells bearing endogenous selectable gene. C. A general principle of generating an endogenous selectable gene for use in co-editing experiments. In this scheme, CRISPR is used to target for example TAF1 gene for mutating it to temperature sensitive. Once obtaining such a temperature sensitive (ts) cell line, it can be used for co-editing to make it temperature resistant (gRNA1 tr) with high efficiency of editing a second gene of interest. Using such a protocol, colonies are obtained overriding the need for single cell culturing. D. Using a drug resistant mutation (dr) for coediting. This is considered semi-scarless since the cells carry a drug resistant mutation.

FIG. 2. Editing of HEK293 to produce TAF1 G716D ts cells. Targeted locus of human TAF1. Guide sequences for targeting the wild-type (wt) locus (GGA CCC TTA ATG ATG CAG GTT GGC ATG GCA—SEQ ID NO: 36), and subsequently the mutated locus, are indicated in green, and the PAM sequences are in pink. The ts mutation (GGA CCC TTA ATG ATG CAG GTT GaC ATG GCA—SEQ ID NO: 37) which converts glycine 716 to aspartic acid 716, creates a HincII cleavage site that was used for selection of positive colonies and editing validation was performed by sequencing.

FIGS. 3A-C. Co-editing efficiency based on ts selection. A. Schematic representation of the targeted PSMB6 locus and donor plasmid. The nucleic acid sequence targeted in PSMB6 is TCGCCGTTGCCACTTTACCACCCGCCTGAATCCTGGGATTCTAGTATGCAA (SEQ ID NO: 38). The structure of the PSMB6 gene is shown with the blue boxes indicating the exons. Stop codon is indicated in red. Guide sequence is in bold, with PAM sequence in pink (note that the guide is the minus strand). The region of the plasmid donor DNA with the 1 kb left and right homology arms (HA) and SYFP insert is shown by the blue lines. The plasmid backbone of the donor DNA is pBluescript KS-. B. Co-editing of HEK293 TAFts to revert ts mutation and to generate the YFP-PSMB6 chimeric protein. HEK293 TAFts cells were transfected with donor DNA for ts reversion to wt and plasmid donor shown in FIG. 3A to coedit in generating the chimeric PSMB6-YFP together with Cas9/guide RNA in targeting TAF1 and PSMB6 for co-editing, or with non-specific sgRNAs (control). Transfected cells were re-plated into 6 cm dishes and incubated at 39.5° C., to exclude the ts cells. Colonies were photographed 20 days later using the ImageQuant LAS 4000 system, using both the Cy3 filter to view YFP fluorescence, and brightfield. C. Cells were plated at 37° C. (no selection) or at 39.5° C. (high temp selection) and pools of cells were analyzed by Western blotting. Dilutions of the selected cell extract were compared to the controls, showing a 50-fold enrichment of PSMB6-YFP co-editing (red arrows).

FIGS. 4A-C. Editing of HEK293 to produce RPL36A P54Q cells. Targeted locus of human RPL36A (ACT AAG CCG ATT TTC CGG AAA AAG GTGAGTGGT—SEQ ID NO: 39). Guide sequence for targeting the wt locus is indicated in green, and the PAM sequence in pink. B, C. HEK293 cells were transfected with donor DNA for both RPL36A P54Q (a mutation conferring resistance to cycloheximide) and for co-editing process to generate the PSMB6-YFP chimeric protein, and Cas9/guide plasmids as indicated. Following transfection, cells were re-plated, and treated with cycloheximide. B. The obtained individual cycloheximide-resistant clones were isolated and analyzed by Western blotting using anti-YFP antibody. The co-edited YFP-expressing clones express the 50-kDa PSMB6-YFP protein. C. Surviving cells formed colonies, which were photographed with the Cy3 filter to show YFP expression; and brightfield, to quantify total colony numbers and co-editing efficiency.

FIGS. 5A-B. An exemplary DHFR mutation in human—GGA GAC CTA CCC TGG CCT CCG CTC AGG TATC (SEQ ID NO: 40) (FIG. 5A) and mouse—GGA GAC CTA CCC TGG CCT CCG CTC AGG TATT—(SEQ ID NO: 41) (FIG. 5B) that renders the cells Methotrexate resistant. Guide sequence for targeting the wt locus is indicated in green, and the PAM sequence in pink.

FIG. 6. Targeted locus of human R2—TTT ATA TCC CAT GTT CTG GCT TTC TTT GCA GCA (SEQ ID NO: 42). Guide sequence for targeting the wild-type locus is indicated in green, and the PAM sequence in pink.

FIGS. 7A-B. Scarless three step strategy. 1. Cells are transfected with Cas9/sgRNA and donor DNA for editing the gene of interest, plus sgRNA/donor for creating the ts mutation (ts), and for creating the drug-resistance mutation (dr). 2. The drug resistant colonies are selected for colonies that do not grow at high temperature but only at low temperature. 3. Colonies are transfected with Cas9/sgRNA and donor for correcting the drug resistant mutation back to wild type. At this stage cells are ts and carry the desired editing. 4. Cells are transfected with Cas9/sgRNA and donor for correcting the ts mutation back to wt. At this stage the obtained colonies are wild type except that they carry the edited gene of interest (FIG. 7A). In the second protocol steps 3 and 4 are combined (FIG. 7B).

FIG. 8A-C illustrates steps required to carry out a siRNA/RNAi/shRNA mediated co-editing strategy, according to embodiments described herein. Red letters show the introduced mutations in order to escape the siRNA. (FIG. 8B—edited sequence CTTCTTCTACTTCTACTACTACTT—(SEQ ID NO: 43); WT sequence CTTCTACTGCTCCTCCTACTACTT—(SEQ ID NO: 44).

FIG. 9. siRNA/RNAi/shRNA mediated co-editing strategy, according to embodiments described herein. The figure provides an exemplary sequence of donor ssODN that can be used to generate a PSMD1 gene sequence that can escape an siRNA agent (e.g. having the sequence TAAGCATTCCCAATATGAG—SEQ ID NO: 50). Highlighted in yellow, is the region targeted by siRNA. The blue box highlights a generated EcoR1 site for sequence confirmation. Red letters show the introduced mutations in order to escape the siRNA. Wild-type PSMA1 3′ UTR (capital letters) tcttccctttcccagGATCTCACTTGCTTATCTGAAGAAGATTGTCCAGGCTCATATTGGGAATG CTTATGAGGAAATTCATGCCGAGACCTGCTATTCAATGCATGTATCGTTGCCTC (SEQ ID NO: 45). The donor ssODN

(SEQ ID NO: 46)
tcttccctttcccagGATCTCACTTGCTTATCTGAAGAAGATTGTCCAG
GCTgATAgTctGAATtCaTATGAGGAAATTCATGCCGAGACCTGCTATT
CAATGCATGTATCGTTGCCTC.

FIGS. 10A-D. Editing of PSMB6-YFP is improved with psmd1 siRNA Selection (A) Time-table of treatments and the experiments. B. HeLa cells were transfected with Cas9/sgRNA encoding plasmids and with donor DNA. Next, cells were transfected with siRNA against psmd1 or luciferase as a control once (+) or twice (++, two rounds of treatments). (C) The surviving cells were expanded and analyzed by FACS with 30,000 cells per point (D) and by SDS-PAGE followed by immunoblotting with the indicated antibodies. The experiment shows that the co-edited protocol is more efficient (17% vs 5%). The treatment twice with siRNA improves efficiency (37% vs 17%).

FIGS. 11A-F illustrate correction of TAF1ts mutation by co-editing using the psmd1 siRNA selection in HEK293 cells. A: guide and ssODN targeting the mutant TAF1, to correct to wt. humTAF1_ts-mut_g2_ CTTAATGATGCAGGTTGaCATGG—the PAM sequence TGG is issustrated in red—SEQ ID NO: 48; ssODN_humTAFwt (-strand)

(SEQ ID NO: 49)
CTGAGCAGAGACTCACCCGTTTATAATAGTTCTTTATCTTGGTTGCCAT
GCCAACCTGCATCATTAAGGGTCCATTTTCCTCACTATATTCTGCAAGA
AT.

B: protocol of HEK293 TAF1 is cells transfection with Cas9/sgRNA expressing plasmids and the ssODN donors. Next, cells were transfected with siRNA against psmd1 or luciferase as a control. The surviving cells were expanded, replated, and transferred the next day to 39.5° C. One week later, colonies growing at 39.5° C. were stained with crystal violet. C: description of the A to E groups of treatments. D: colonies growing at 39.5° C. were stained with crystal violet. E: For control of plating efficiency, cell density was measured at 37° C. F: numbers of colonies in each group. N=3, *p=0.03. The co-editing selection was shown to be four times more efficient.

### DESCRIPTION OF SPECIFIC EMBODIMENTS OF THE INVENTION

The present invention, in some embodiments thereof, relates to a method of selecting cells which have undergone targeted genome editing event without leaving a selection marker footprint in the cell.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

CRISPR/Cas9 is a powerful tool for genome editing in cells and organisms. Nevertheless, introducing directed templated changes by homology-directed repair (HDR) requires the cellular DNA repair machinery, such as the MRN complex (Mre11/Rad50/Nbs1). HDR normally occurs only during the S/G2/M portion of the cell cycle, and thus in most cases, only a minority of the cells are competent to make templated genomic changes. Therefore, a way to select for these cells is needed. Previous studies have shown that it is possible to edit multiple loci at once using CRISPR. Thus, the present inventors reasoned that if one of the edited sites provides a selectable marker, this can be used to enrich the pool of co-edited cells. Up until presently, insertion of foreign genes encoding fluorescent proteins or proteins that confer antibiotic resistance have been used for such protocols. However, for many applications it is undesirable to introduce a foreign gene.

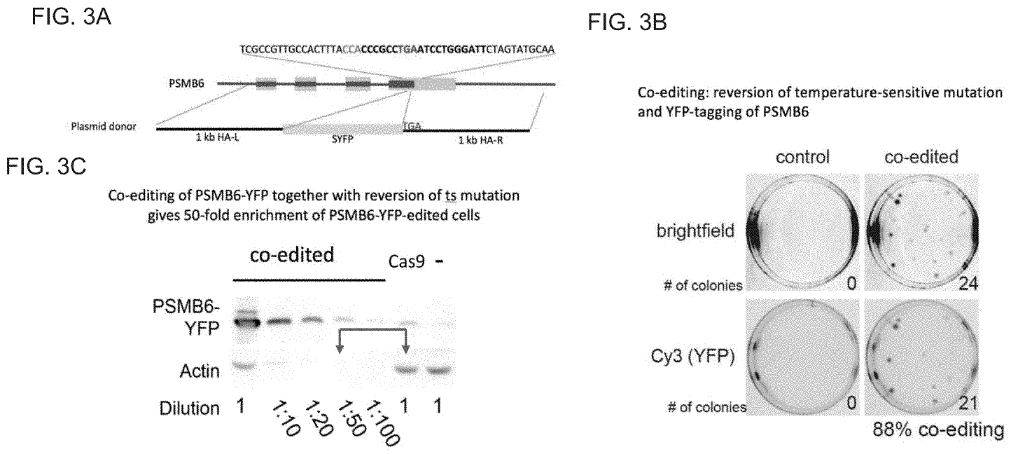

The present inventors have now formulated a strategy for “scarless selection” based on converting an endogenous gene into a selectable marker. As a proof of concept, the present inventors created a temperature-sensitive (ts) cell line with a point mutation in the TAF1 gene. TAFts can be reverted into its native sequence using CRISPR editing. Co-editing of the ts gene in addition to a gene of interest provides individual clones, overriding the need for single cell cloning, with enrichment of up to 90% for the desired editing (see FIGS. 3A-C).

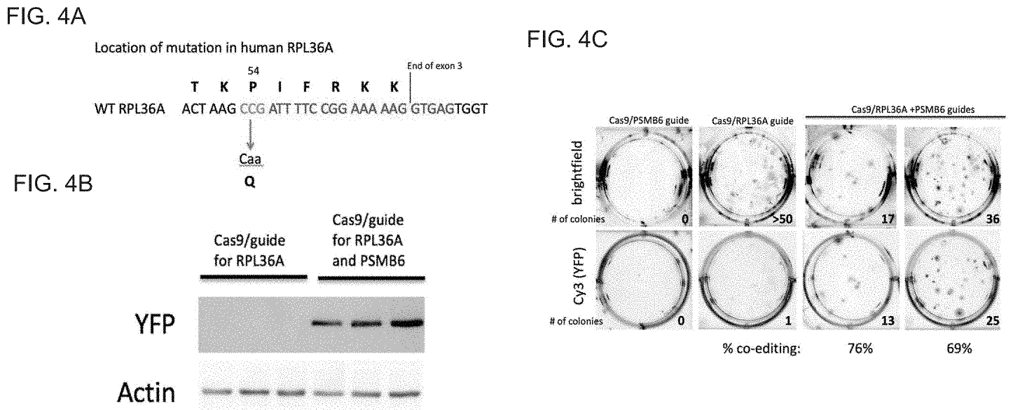

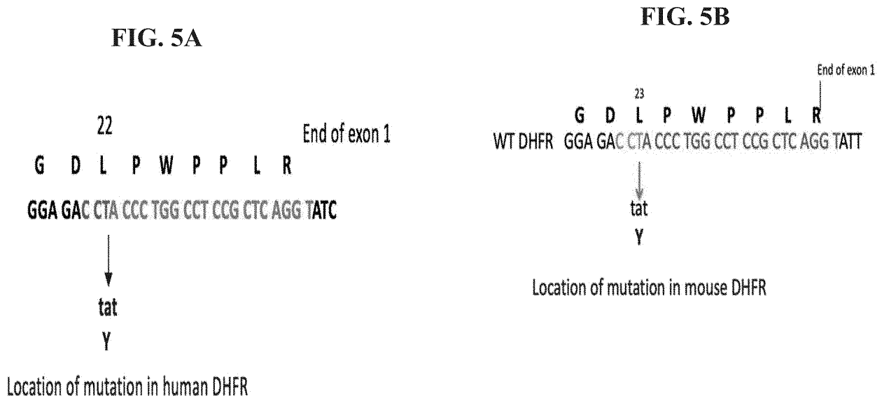

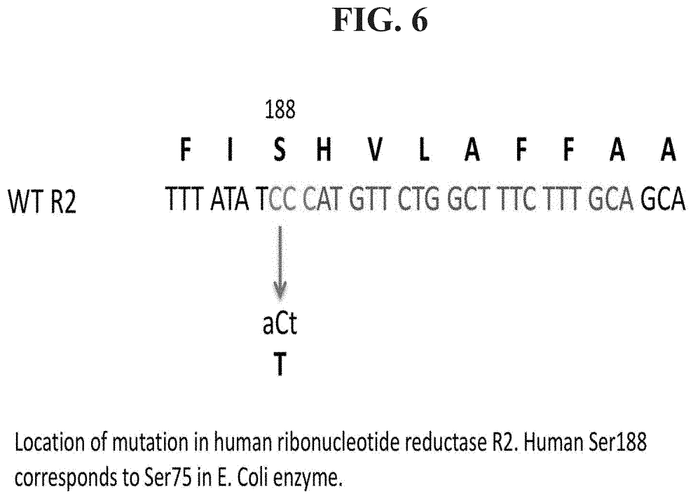

Whilst further reducing the present invention to practice, the present inventors showed that it was possible to convert an endogenous gene into a selectable marker which imparts resistance to a cytotoxic drug by introduction of a single point mutation (see FIGS. 4A-C). The method of selection in this case is virtually scarless as the point mutation has no bearing on the endogenous function of the gene.

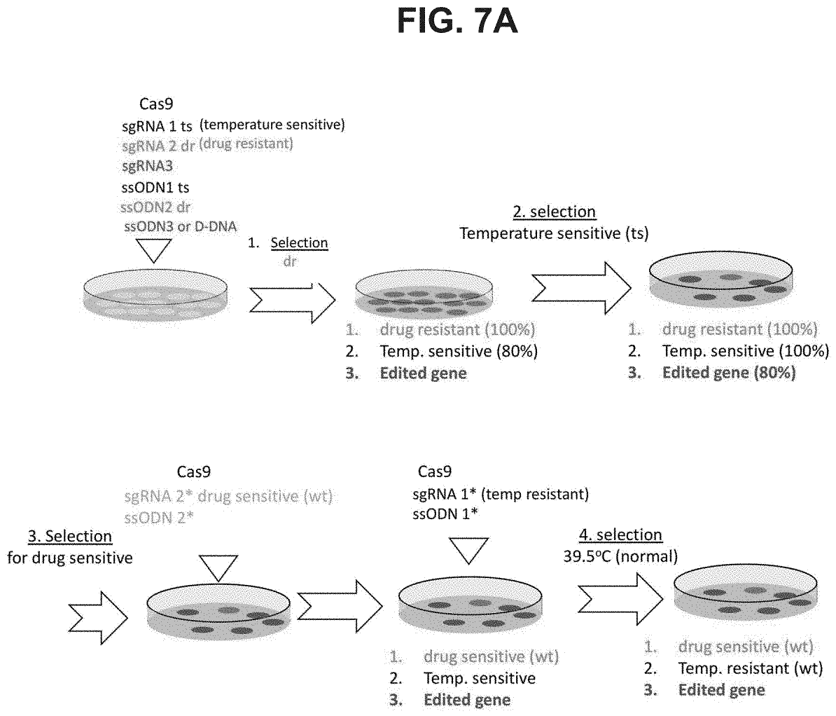

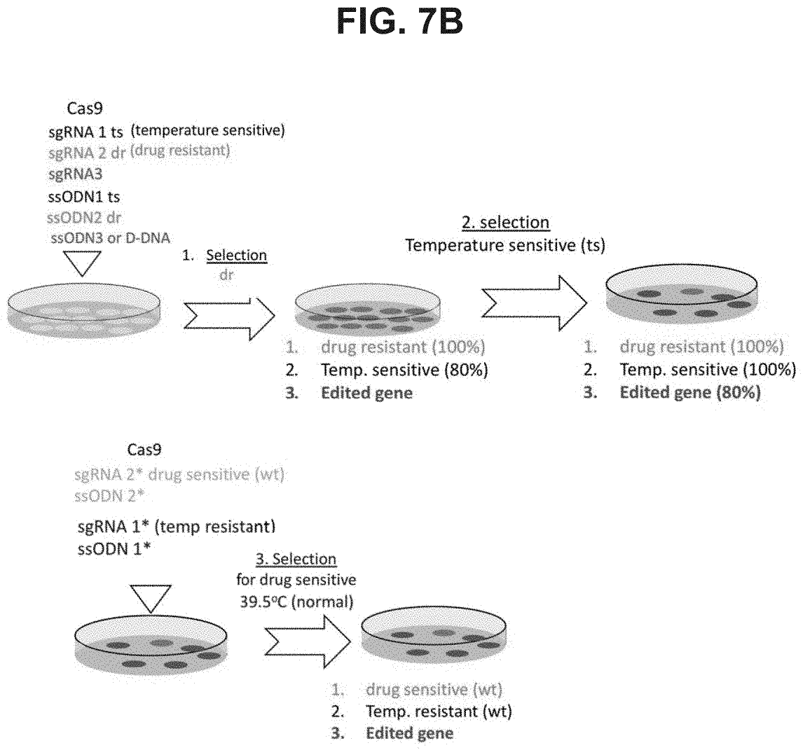

By using two selectable markers in two CRISPR steps, editing of desired genes with fully scarless selection can be achieved in any naive cell (FIG. 7).

Thus, according to a first aspect of the present invention, there is provided a method of selecting a cell which harbors a genome-editing event at a target sequence of interest comprising:
(a) providing cells having a genome, the cells comprising a mutation on a first gene of the genome, the mutation rendering the first gene a selection marker;
(b) co-transfecting the cells with:
   (i) a first DNA editing agent for specifically disrupting selection marker activity of the selection marker; and
   (ii) a second DNA editing agent for specifically editing the genome at the target sequence of interest; and
(c) culturing the cells under conditions that enrich for cells that do not comprise the selection marker, thereby selecting a cell which harbors the genome-editing event.

The term “genome editing” refers to a type of genetic engineering in which DNA is inserted, replaced, or removed from a target DNA, e.g., the genome of a cell, using one or more nucleases and/or nickases.

The nucleases create specific double-strand breaks (DSBs) at desired locations in the genome, and harness the cell's endogenous mechanisms to repair the induced break by homology-directed repair (HDR) (e.g., homologous recombination) or by nonhomologous end joining (NHEJ). The nickases create specific single-strand breaks at desired locations in the genome. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. Further details about genome editing are provided herein below.

According to a particular embodiment, the genome editing event is brought about by homology-directed repair.

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

According to one embodiment, the genome-editing event is a modification selected from the group consisting of a deletion, an insertion, a point mutation and a combination thereof (e.g. insertion-deletion (Indel)).

According to one embodiment, the modification comprises a modification of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 150, 250, 500, 1000, 1500, 2000, 3000, 4000 or at most 5000 nucleotides.

According to one embodiment, the modification comprises a deletion.

According to one embodiment, the deletion comprises a deletion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 150, 250, 500, 1000, 1500, 2000, 3000, 4000 or at most 5000 nucleotides.

According to one embodiment, the deletion comprises a deletion of an entire gene.

According to one embodiment, the modification comprises a point mutation.

According to one embodiment, the point mutation comprises a point mutation in at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 150, 250, 500, 1000, 1500, 2000, 3000, 4000 or at most 5000 nucleotides.

According to one embodiment, the modification comprises an insertion.

According to one embodiment, the insertion comprises an insertion of at most 1, 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, 50, 60, 70, 80, 90, 100, 150, 250, 500, 1000, 1500, 2000, 3000, 4000 or at most 5000 nucleotides.

According to one embodiment, the insertion comprises an insertion of an entire gene.

According to a specific embodiment, the modification is a mutation in the coding sequence of a gene, e.g. changing a wild-type sequence to a mutated sequence.

According to a specific embodiment, the modification is a reversion in the coding sequence of a gene, e.g. reverting a mutant sequence to a wild-type sequence.

According to a specific embodiment, the modification is an addition or deletion of a gene segment, e.g. giving rise to a protein with an added domain (natural or artificial), or a deletion.

According to a specific embodiment, the modification is in a non-coding element of a gene, e.g. creating mutations, deletions, or insertions in non-coding elements, including promoter regions, enhancer regions, transcription start sites, translation start sites, splice sites, introns, terminator regions, 5′ and 3′ UTR regions (of the encoded mRNA). Such edited changes can affect, for example, mRNA expression, stability, splicing, export, translation.

According to a specific embodiment, the modification is an epigenetic modification in the genomic region, with implications for expression of genes at that locus.

According to a specific embodiment, the modification targets non-coding RNAs, including e.g. tRNAs, ribosomal RNAs, other RNAs with non-protein-coding functions.

According to a specific embodiment, the modification targets genes of regulatory non-coding RNAs, such as miRNAs, which can affect for example the target genes of those miRNAs.

According to a specific embodiment, the modification alters miRNA recognition sites of target genes, e.g. changing expression of those genes.

According to a specific embodiment, the modification changes chromatin architecture, for example by targeting CTCF binding sites, with impact on gene expression in the targeted chromatin domain.

The starting cell population of this aspect of the present invention have a mutation on an endogenous gene of the genome thereof, which converts the gene into a selection marker.

Any type of cell population is contemplated for this aspect of the present invention. The cells may be primary cells or may be part of a cell line. In one embodiment, the cell population comprises eukaryotic cells, such as mammalian cells (e.g. human cells). In one embodiment, the cell population is a diseased cell population (e.g. cancer cells). In another embodiment, the cell population comprises healthy cells.

In some embodiments, the cell is isolated from a multicellular organism prior to use. The multicellular organism can be a plant, a multicellular protist, a multicellular fungus, or an animal such as a mammal (e.g., human). In certain instances, the cell is selected from the group consisting of a stem cell, an immune cell, and a combination thereof. Non-limiting examples of stem cells include embryonic stem cells, induced pluripotent stem cells, hematopoietic stem and progenitor cells (HSPCs) such as CD34+ HSPCs, mesenchymal stem cells, neural stem cells, organ stem cells, and combinations thereof. Non-limiting examples of immune cells include T cells (e.g., CD3+ T cells, CD4+ T cells, CD8+ T cells, tumor infiltrating cells (TILs), memory T cells, memory stem T cells, effector T cells), natural killer cells, monocytes, peripheral blood mononuclear cells (PBMCs), peripheral blood lymphocytes (PBLs), and combinations thereof.

Additional exemplary cell populations include, but are not limited to cardiac cells, muscle cells, skin cells, pancreatic cells, liver cells, glial cells, lung cells and kidney cells.

Any type of mutation is contemplated that converts a gene of the genome into a selection marker. Thus, for example point mutations, insertion and deletions are all contemplated.

According to a specific embodiment, the mutation is a point mutation—i.e. a single amino acid substitution.

Methods of introducing mutations into a gene of the genome of the cell are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination, site specific recombinases, PB transposases and genome editing by engineered nucleases. Agents for introducing nucleic acid alterations to a gene of interest can be designed publically available sources or obtained commercially from Transposagen, Addgene and Sangamo Biosciences.

Following is a description of various exemplary methods used to introduce nucleic acid alterations to a gene of interest and agents for implementing same that can be used according to specific embodiments of the present invention.

Genome Editing using engineered endonucleases—this approach refers to a reverse genetics method using artificially engineered nucleases to cut and create specific double-stranded breaks at a desired location(s) in the genome, which are then repaired by cellular endogenous processes such as, homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In order to introduce specific nucleotide modifications to the genomic DNA, a DNA repair template containing the desired sequence must be present during HDR. Genome editing cannot be performed using traditional restriction endonucleases since most restriction enzymes recognize a few base pairs on the DNA as their target and the probability is very high that the recognized base pair combination will be found in many locations across the genome resulting in multiple cuts not limited to a desired location. To overcome this challenge and create site-specific single- or double-stranded breaks, several distinct classes of nucleases have been discovered and bioengineered to date. These include the meganucleases, Zinc finger nucleases (ZFNs), transcription-activator like effector nucleases (TALENs) and CRISPR/Cas system.

Meganucleases—Meganucleases are commonly grouped into four families: the LAGLIDADG (SEQ ID NO: 20) family, the GIY-YIG (SEQ ID NO: 21) family, the His-Cys box family and the HNH family. These families are characterized by structural motifs, which affect catalytic activity and recognition sequence. For instance, members of the LAGLIDADG (SEQ ID NO: 20) family are characterized by having either one or two copies of the conserved LAGLIDADG (SEQ ID NO: 20) motif. The four families of meganucleases are widely separated from one another with respect to conserved structural elements and, consequently, DNA recognition sequence specificity and catalytic activity. Meganucleases are found commonly in microbial species and have the unique property of having very long recognition sequences (>14 bp) thus making them naturally very specific for cutting at a desired location. This can be exploited to make site-specific double-stranded breaks in genome editing. One of skill in the art can use these naturally occurring meganucleases, however the number of such naturally occurring meganucleases is limited. To overcome this challenge, mutagenesis and high throughput screening methods have been used to create meganuclease variants that recognize unique sequences. For example, various meganucleases have been fused to create hybrid enzymes that recognize a new sequence. Alternatively, DNA interacting amino acids of the meganuclease can be altered to design sequence specific meganucleases (see e.g., U.S. Pat. No. 8,021,867). Meganucleases can be designed using the methods described in e.g., Certo, M T et al. Nature Methods (2012) 9:073-975; U.S. Pat. Nos. 8,304,222; 8,021,867; 8,119,381; 8,124,369; 8, 129,134; 8,133,697; 8,143,015; 8,143,016; 8, 148,098; or 8, 163,514, the contents of each are incorporated herein by reference in their entirety. Alternatively, meganucleases with site specific cutting characteristics can be obtained using commercially available technologies e.g., Precision Biosciences' Directed Nuclease Editor™ genome editing technology.

ZFNs and TALENs—Two distinct classes of engineered nucleases, zinc-finger nucleases (ZFNs) and transcription activator-like effector nucleases (TALENs), have both proven to be effective at producing targeted double-stranded breaks (Christian et al., 2010; Kim et al., 1996; Li et al., 2011; Mahfouz et al., 2011; Miller et al., 2010).

Basically, ZFNs and TALENs restriction endonuclease technology utilizes a non-specific DNA cutting enzyme which is linked to a specific DNA binding domain (either a series of zinc finger domains or TALE repeats, respectively). Typically a restriction enzyme whose DNA recognition site and cleaving site are separate from each other is selected. The cleaving portion is separated and then linked to a DNA binding domain, thereby yielding an endonuclease with very high specificity for a desired sequence. An exemplary restriction enzyme with such properties is Fok1. Additionally Fok1 has the advantage of requiring dimerization to have nuclease activity and this means the specificity increases dramatically as each nuclease partner recognizes a unique DNA sequence. To enhance this effect, Fok1 nucleases have been engineered that can only function as heterodimers and have increased catalytic activity. The heterodimer functioning nucleases avoid the possibility of unwanted homodimer activity and thus increase specificity of the double-stranded break.

Thus, for example to target a specific site, ZFNs and TALENs are constructed as nuclease pairs, with each member of the pair designed to bind adjacent sequences at the targeted site. Upon transient expression in cells, the nucleases bind to their target sites and the Fok1 domains heterodimerize to create a double-stranded break. Repair of these double-stranded breaks through the nonhomologous end-joining (NHEJ) pathway most often results in small deletions or small sequence insertions. Since each repair made by NHEJ is unique, the use of a single nuclease pair can produce an allelic series with a range of different deletions at the target site. The deletions typically range anywhere from a few base pairs to a few hundred base pairs in length, but larger deletions have successfully been generated in cell culture by using two pairs of nucleases simultaneously (Carlson et al., 2012; Lee et al., 2010). In addition, when a fragment of DNA with homology to the targeted region is introduced in conjunction with the nuclease pair, the double-stranded break can be repaired via homology directed repair to generate specific modifications (Li et al., 2011; Miller et al., 2010; Urnov et al., 2005).

Although the nuclease portions of both ZFNs and TALENs have similar properties, the difference between these engineered nucleases is in their DNA recognition peptide. ZFNs rely on Cys2-His2 zinc fingers and TALENs on TALEs. Both of these DNA recognizing peptide domains have the characteristic that they are naturally found in combinations in their proteins. Cys2-His2 Zinc fingers are typically found in repeats that are 3 bp apart and are found in diverse combinations in a variety of nucleic acid interacting proteins. TALEs on the other hand are found in repeats with a one-to-one recognition ratio between the amino acids and the recognized nucleotide pairs. Because both zinc fingers and TALEs happen in repeated patterns, different combinations can be tried to create a wide variety of sequence specificities. Approaches for making site-specific zinc finger endonucleases include, e.g., modular assembly (where Zinc fingers correlated with a triplet sequence are attached in a row to cover the required sequence), OPEN (low-stringency selection of peptide domains vs. triplet nucleotides followed by high-stringency selections of peptide combination vs. the final target in bacterial systems), and bacterial one-hybrid screening of zinc finger libraries, among others. ZFNs can also be designed and obtained commercially from e.g., Sangamo Biosciences™ (Richmond, CA).

Method for designing and obtaining TALENs are described in e.g. Reyon et al. Nature Biotechnology 2012 May; 30(5):460-5; Miller et al. Nat Biotechnol. (2011) 29: 143-148; Cermak et al. Nucleic Acids Research (2011) 39 (12): e82 and Zhang et al. Nature Biotechnology (2011) 29 (2): 149-53. A recently developed web-based program named Mojo Hand was introduced by Mayo Clinic for designing TAL and TALEN constructs for genome editing applications (can be accessed through www(dot)talendesign(dot)org). TALEN can also be designed and obtained commercially from e.g., Sangamo Biosciences™ (Richmond, CA).

CRISPR-Cas system—The CRISPR/Cas system of genome modification includes a Cas nuclease (e.g., Cas9 nuclease) or a variant or fragment thereof, a DNA-targeting RNA (e.g., single guide RNA (sgRNA)) containing a guide sequence that targets the Cas nuclease to the target genomic DNA and a scaffold sequence that interacts with the Cas nuclease (e.g., tracrRNA), and optionally, a donor repair template. In some instances, a variant of a Cas nuclease such as a Cas9 mutant containing one or more of the following mutations: D10A, H840A, D839A, and H863A, or a Cas9 nickase can be used. In other instances, a fragment of a Cas nuclease or a variant thereof with desired properties (e.g., capable of generating single- or double-strand breaks and/or modulating gene expression) can be used. The donor repair template can include homology arms that are homologous to the target DNA and flank the site of gene modification. The donor repair template can be provided on a ds plasmid or via a viral vector or as a ds linear fragment. Alternatively, the donor repair template can be a single-stranded oligodeoxynucleotide (ssODN).

1. Cas Nucleases and Variants Thereof:

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease requires both the crRNA and the tracrRNA for site-specific DNA recognition and cleavage. This system has now been engineered such that the crRNA and tracrRNA can be combined into one molecule (the “single guide RNA” or “sgRNA”), and the crRNA equivalent portion of the single guide RNA can be engineered to guide the Cas (e.g., Cas9) nuclease to target any desired sequence (see, e.g., Jinek et al. (2012) Science, 337:816-821; Jinek et al. (2013) eLife, 2:e00471; Segal (2013) eLife, 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

In some embodiments, the Cas nuclease has DNA cleavage activity. The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, variants thereof, fragments thereof, mutants thereof, and derivatives thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas 1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. CRISPR-related endonucleases that are useful in the present invention are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cas9” refers to an RNA-guided double-stranded DNA-binding nuclease protein or nickase protein. Wild-type Cas9 nuclease has two functional domains, e.g., RuvC and HNH, that cut different DNA strands. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single-strand break or nick. In some embodiments, the mutant Cas9 nuclease having at least a D10A mutation is a Cas9 nickase. In other embodiments, the mutant Cas9 nuclease having at least a H840A mutation is a Cas9 nickase. Other examples of mutations present in a Cas9 nickase include, without limitation, N854A and N863A. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of indel mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the target cell or target organism.

In some embodiments, the Cas nuclease can be a Cas9 polypeptide that contains two silencing mutations of the RuvC1 and HNH nuclease domains (D10A and H840A), which is referred to as dCas9 (Jinek et al., Science, 2012, 337:816-821; Qi et al., Cell, 152(5):1173-1183). In one embodiment, the dCas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The dCas9 enzyme can contain a mutation at D10, E762, H983 or D986, as well as a mutation at H840 or N863. In some instances, the dCas9 enzyme contains a D10A or D10N mutation. Also, the dCas9 enzyme can include a H840A, H840Y, or H840N. In some embodiments, the dCas9 enzyme of the present invention comprises D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

For genome editing methods, the Cas nuclease can be a Cas9 fusion protein such as a polypeptide comprising the catalytic domain of the type IIS restriction enzyme, FokI, linked to dCas9. The FokI-dCas9 fusion protein (fCas9) can use two guide RNAs to bind to a single strand of target DNA to generate a double-strand break.

In some embodiments, a nucleotide sequence encoding the Cas nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Depending on the target cell/expression system used, any of a number of transcription and translation control elements, including promoter, transcription enhancers, transcription terminators, and the like, may be used in the expression vector. Useful promoters can be derived from viruses, or any organism, e.g., prokaryotic or eukaryotic organisms. Suitable promoters include, but are not limited to, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc.

The Cas nuclease and variants or fragments thereof can be introduced into a cell (e.g., an in vitro cell such as a primary cell for ex vivo therapy, or an in vivo cell such as in a patient) as a Cas polypeptide or a variant or fragment thereof, an mRNA encoding a Cas polypeptide or a variant or fragment thereof, or a recombinant expression vector comprising a nucleotide sequence encoding a Cas polypeptide or a variant or fragment thereof.

2. Guide RNA (gRNA)

The gRNAs for use in the CRISPR/Cas system of genome modification typically include a guide sequence (e.g., crRNA) that is complementary to a target nucleic acid sequence and a scaffold sequence (e.g., tracrRNA) that interacts with a Cas nuclease (e.g., Cas9 polypeptide) or a variant or fragment thereof.

The gRNA may be modified such that it comprises modified nucleotides as further described in US Patent No. 20180119140, the contents of which are incorporated herein by reference. In certain instances, the gRNA is complexed with a Cas nuclease (e.g., Cas9 polypeptide) or a variant or fragment thereof to form a ribonucleoprotein (RNP)-based delivery system for introduction into a cell (e.g., an in vitro cell such as a primary cell for ex vivo therapy, or an in vivo cell such as in a patient). In other instances, the gRNA is introduced into a cell with an mRNA encoding a Cas nuclease (e.g., Cas9 polypeptide) or a variant or fragment thereof. In yet other instances, the gRNA is introduced into a cell with a recombinant expression vector comprising a nucleotide sequence encoding a Cas nuclease (e.g., Cas9 polypeptide) or a variant or fragment thereof.

In some instances, a plurality of gRNAs can be used for efficient multiplexed CRISPR-based gene regulation (e.g., genome editing or modulating gene expression) in target cells such as primary cells. The plurality of gRNAs can include at least about 2, 3, 4, 5, 6, 7, 8, 9, 10, 15, 20, 25, 30, 35, 40, 45, 50, or more gRNAs that hybridize to the same target nucleic acid sequence or to different target nucleic acid sequences. The plurality of gRNAs can be introduced into a cell in a complex with a Cas nuclease (e.g., Cas9 polypeptide) or a variant or fragment thereof, or as a nucleotide sequence (e.g., mRNA or recombinant expression vector) encoding a Cas nuclease (e.g., Cas9 polypeptide) or a variant or fragment thereof.

The nucleic acid sequence of the modified gRNA can be any polynucleotide sequence having sufficient complementarity with a target polynucleotide sequence (e.g., target DNA sequence) to hybridize with the target sequence and direct sequence-specific binding of a CRISPR complex to the target sequence. In some embodiments, the degree of complementarity between a guide sequence of the gRNA and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a guide sequence is about 20 nucleotides in length. In other instances, a guide sequence is about nucleotides in length. In other instances, a guide sequence is about 25 nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

The nucleotide sequence of a gRNA can be selected using any of the web-based software described above. Considerations for selecting a DNA-targeting RNA include the PAM sequence for the Cas nuclease (e.g., Cas9 polypeptide) to be used, and strategies for minimizing off-target modifications. Tools, such as the CRISPR Design Tool, can provide sequences for preparing the modified gRNA, for assessing target modification efficiency, and/or assessing cleavage at off-target sites. Another consideration for selecting the sequence of a gRNA includes reducing the degree of secondary structure within the guide sequence. Secondary structure may be determined by any suitable polynucleotide folding algorithm. Some programs are based on calculating the minimal Gibbs free energy. Examples of suitable algorithms include mFold (Zuker and Stiegler, Nucleic Acids Res, 9 (1981), 133-148), UNAFold package (Markham et al., Methods Mol Biol, 2008, 453:3-31) and RNAfold form the ViennaRNa Package.

In the CRISPR/Cas system, the target DNA sequence can be complementary to a fragment of a DNA-targeting RNA (e.g., gRNA) and can be immediately followed by a protospacer adjacent motif (PAM) sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT (SEQ ID NO: 22) the PAM sequence of Streptococcus thermophilus-derived Cas9 is NNAGAA (SEQ ID NO: 23); and the PAM sequence of Treponema denticola-derived Cas9 is NAAAAC (SEQ ID NO: 24). In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (e.g., be located 5′) a 5′NGG PAM, wherein N is any nucleotide, such that the guide sequence of the DNA-targeting RNA (e.g., gRNA) base pairs with the opposite strand to mediate cleavage at about 3 base pairs upstream of the PAM sequence.

In some embodiments, the degree of complementarity between a guide sequence of the DNA-targeting RNA (e.g., gRNA) and its corresponding target DNA sequence, when optimally aligned using a suitable alignment algorithm, is about or more than about 50%, 55%, 60%, 65%, 70%, 75%, 80%, 85%, 90%, 91%, 92%, 93%, 94%, 95%, 96%, 97%, 98%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, Selangor, Malaysia), and ELAND (Illumina, San Diego, Calif.).

The target DNA site can be selected in a predefined genomic sequence (gene) using web-based software such as ZiFiT Targeter software (Sander et al., 2007, Nucleic Acids Res, 35:599-605; Sander et al., 2010, Nucleic Acids Res, 38:462-468), E-CRISP (Heigwer et al., 2014, Nat Methods, 11:122-123), RGEN Tools (Bae et al., 2014, Bioinformatics, 30(10):1473-1475), CasFinder (Aach et al., 2014, bioRxiv), DNA2.0 gNRA Design Tool (DNA2.0, Menlo Park, Calif.), and the CRISPR Design Tool (Broad Institute, Cambridge, Mass.). Such tools analyze a genomic sequence (e.g., gene or locus of interest) and identify suitable target site for gene editing. To assess off-target gene modifications for each DNA-targeting RNA (e.g., gRNA), computationally predictions of off-target sites are made based on quantitative specificity analysis of base-pairing mismatch identity, position and distribution.

Site-Specific Recombinases—The Cre recombinase derived from the P1 bacteriophage and F1p recombinase derived from the yeast Saccharomyces cerevisiae are site-specific DNA recombinases each recognizing a unique 34 base pair DNA sequence (termed “Lox” and “FRY”, respectively) and sequences that are flanked with either Lox sites or FRT sites can be readily removed via site-specific recombination upon expression of Cre or F1p recombinase, respectively. For example, the Lox sequence is composed of an asymmetric eight base pair spacer region flanked by 13 base pair inverted repeats. Cre recombines the 34 base pair lox DNA sequence by binding to the 13 base pair inverted repeats and catalyzing strand cleavage and religation within the spacer region. The staggered DNA cuts made by Cre in the spacer region are separated by 6 base pairs to give an overlap region that acts as a homology sensor to ensure that only recombination sites having the same overlap region recombine.

Basically, the site specific recombinase system offers means for the removal of selection cassettes after homologous recombination. This system also allows for the generation of conditional altered alleles that can be inactivated or activated in a temporal or tissue-specific manner. Of note, the Cre and F1p recombinases leave behind a Lox or FRT “scar” of 34 base pairs. The Lox or FRT sites that remain are typically left behind in an intron or 3′ UTR of the modified locus, and current evidence suggests that these sites usually do not interfere significantly with gene function.

Thus, Cre/Lox and F1p/FRT recombination involves introduction of a targeting vector with 3′ and 5′ homology arms containing the mutation of interest, two Lox or FRT sequences and typically a selectable cassette placed between the two Lox or FRT sequences. Positive selection is applied and homologous recombinants that contain targeted mutation are identified. Transient expression of Cre or F1p in conjunction with negative selection results in the excision of the selection cassette and selects for cells where the cassette has been lost. The final targeted allele contains the Lox or FRT scar of exogenous sequences.

Transposases—As used herein, the term “transposase” refers to an enzyme that binds to the ends of a transposon and catalyzes the movement of the transposon to another part of the genome.

As used herein the term “transposon” refers to a mobile genetic element comprising a nucleotide sequence which can move around to different positions within the genome of a single cell. In the process the transposon can cause mutations and/or change the amount of a DNA in the genome of the cell.

A number of transposon systems that are able to also transpose in cells e.g. vertebrates have been isolated or designed, such as Sleeping Beauty [Izsvák and Ivics Molecular Therapy (2004) 9, 147-156], piggyBac [Wilson et al. Molecular Therapy (2007) 15, 139-145], To12 [Kawakami et al. PNAS (2000) 97 (21): 11403-11408] or Frog Prince [Miskey et al. Nucleic Acids Res. Dec. 1, (2003) 31(23): 6873-6881]. Generally, DNA transposons translocate from one DNA site to another in a simple, cut-and-paste manner. Each of these elements has their own advantages, for example, Sleeping Beauty is particularly useful in region-specific mutagenesis, whereas To12 has the highest tendency to integrate into expressed genes. Hyperactive systems are available for Sleeping Beauty and piggyBac. Most importantly, these transposons have distinct target site preferences, and can therefore introduce sequence alterations in overlapping, but distinct sets of genes. Therefore, to achieve the best possible coverage of genes, the use of more than one element is particularly preferred. The basic mechanism is shared between the different transposases, therefore we will describe piggyBac (PB) as an example.

PB is a 2.5 kb insect transposon originally isolated from the cabbage looper moth, Trichoplusia ni. The PB transposon consists of asymmetric terminal repeat sequences that flank a transposase, PBase. PBase recognizes the terminal repeats and induces transposition via a “cut-and-paste” based mechanism, and preferentially transposes into the host genome at the tetranucleotide sequence TTAA. Upon insertion, the TTAA target site is duplicated such that the PB transposon is flanked by this tetranucleotide sequence. When mobilized, PB typically excises itself precisely to reestablish a single TTAA site, thereby restoring the host sequence to its pretransposon state. After excision, PB can transpose into a new location or be permanently lost from the genome.

Typically, the transposase system offers an alternative means for the removal of selection cassettes after homologous recombination quit similar to the use Cre/Lox or F1p/FRT. Thus, for example, the PB transposase system involves introduction of a targeting vector with 3′ and 5′ homology arms containing the mutation of interest, two PB terminal repeat sequences at the site of an endogenous TTAA sequence and a selection cassette placed between PB terminal repeat sequences. Positive selection is applied and homologous recombinants that contain targeted mutation are identified. Transient expression of PBase removes in conjunction with negative selection results in the excision of the selection cassette and selects for cells where the cassette has been lost. The final targeted allele contains the introduced mutation with no exogenous sequences.

For PB to be useful for the introduction of sequence alterations, there must be a native TTAA site in relatively close proximity to the location where a particular mutation is to be inserted.

It will be appreciated that an agent may be used that causes random mutations and the cells having the desired marker activity may be selected.

The mutagens may be, but are not limited to, genetic, chemical or radiation agents. For example, the mutagen may be ionizing radiation, such as, but not limited to, ultraviolet light, gamma rays or alpha particles. Other mutagens may include, but not be limited to, base analogs, which can cause copying errors; deaminating agents, such as nitrous acid; intercalating agents, such as ethidium bromide; alkylating agents, such as bromouracil; transposons; natural and synthetic alkaloids; bromine and derivatives thereof; sodium azide; psoralen (for example, combined with ultraviolet radiation). The mutagen may be a chemical mutagen such as, but not limited to, ICR191, 1,2,7,8-diepoxy-octane (DEO), 5-azaC, N-methyl-N-nitrosoguanidine (MNNG) or ethyl methane sulfonate (EMS).

Together with the endonuclease, the present inventors further contemplate using a polypeptide capable of increasing homology-directed repair. Such polypeptides are described in PCT application No. IL2019/050707, the contents of which are incorporated herein by reference and summarized herein below

According to one embodiment, the polypeptide capable of increasing homologous recombination is capable of recruiting at least one of MRN/ATM-dependent DNA damage response factors (e.g. γH2AX, Chk2, 53BP1, Rad17, MRN complex [Mre11, Rad50, Nbs1], MDC1, CtIP, ATR, ATRIP, TopBP1, 9-1-1 complex (Rad9, HUS1, Rad1)), homologous recombination proteins (e.g. Rad51, Rad52, Rad53, Rad54, Rad55/57, Shu complex i.e. Shu1, Psy3, Shu2 and Csm2 proteins, BRCA2, BARD1, and BRCA1) or DNA-dependent ATPases associated with homologous recombination (e.g. Snf2/Swi2).

According to one embodiment, the polypeptide capable of increasing homologous recombination is capable of recruiting at least one component of the cellular MRN complex (Mre11/Rad50/Nbs1).

According to one embodiment, the polypeptide capable of increasing homologous recombination is an alkaline nuclease.

According to one embodiment, the polypeptide capable of increasing homologous recombination comprises the YqaJ conserved protein domain (also known as the YqaJ-like viral recombinase domain).

According to one embodiment, the polypeptide capable of increasing homologous recombination is a viral polypeptide (e.g. a viral alkaline nuclease, a viral DNase, or a viral alkaline exonuclease) or fragment thereof capable of recruiting at least one component of the cellular MRN complex (Mre11/Rad50/Nbs1).

According to one embodiment, the viral peptide is derived from a herpesvirus.

Exemplary herpesviruses from which the viral peptide may be derived include, but are not limited to, Herpes simplex virus 1 (HSV-1), Herpes simplex virus 2 (HSV-2), Varicella zoster virus (VZV), Epstein-Barr virus (EBV), Cytomegalovirus (CMV), Roseolovirus, Kaposi's sarcoma-associated herpesvirus (KSHV), Pseudorabies virus (PRV), and Bovine herpesvirus.

According to a specific embodiment, the viral peptide is derived from HSV-1.

According to a specific embodiment, the viral peptide is UL12, a homolog or a fragment thereof. An exemplary UL12 is set forth in SEQ ID NO: 25.

According to a specific embodiment, the UL12 comprises amino acids 1-126 of an N-terminal fragment of UL12, e.g. as set forth in SEQ ID NO: 26, a homolog or a fragment thereof.

According to one embodiment, the UL12 comprises an amino acid sequence at least 90%, 95%, 99%, 100% identical to SEQ ID NO: 26.

According to one embodiment, the UL12 comprises a fragment of 50-126, 60-126, 70-126, 80-126, 90-126, 100-126, 110-126 or 120-126 amino acids of the amino acid sequence set forth in SEQ ID NO: 26.

According to one embodiment, the UL12 comprises a fragment of 50, 60, 70, 80, 90, 100, 110, 120, 121, 122, 123, 124 or 125 consecutive amino acids of the amino acid sequence set forth in SEQ ID NO: 26.

According to a specific embodiment, the UL12 comprises amino acids 50-126 of an N-terminal fragment of UL12.

According to a specific embodiment, the viral peptide is derived from HSV-2, e.g. UL12, a homolog or a fragment thereof.

According to a specific embodiment, the viral peptide is derived from Bovine herpesvirus, e.g. UL12, a homolog or a fragment thereof.

According to one embodiment, the viral peptide is derived from CMV, e.g. UL98, a homolog or a fragment thereof.

According to one embodiment, the viral peptide is derived from EBV, e.g. P03217 (AN_EBVB9), a homolog or a fragment thereof.

According to one embodiment, the viral peptide is derived from VZV, e.g. ORF48, a homolog or a fragment thereof.

According to one embodiment, the viral peptide is derived from a baculovirus, e.g. alkaline nuclease, a homolog or a fragment thereof.

According to a specific embodiment, the viral peptide is derived from baculovirus Autographa californica multinucleocapsid nucleopolyhedrovirus (AcMNPV) open reading frame 133.

According to one embodiment, the viral peptide is the plant virus protein At3g48810, a homolog or a fragment thereof.

According to one embodiment, the UL12 homolog is the protein of unknown function DUF3292 (IPR021709).

According to one embodiment, the polypeptide capable of increasing homologous recombination is a eukaryotic polypeptide or fragment thereof capable of recruiting at least one component of the cellular MRN complex (Mre11/Rad50/Nbs1).

According to one embodiment, the polypeptide capable of increasing homologous recombination is a eukaryotic polypeptide or fragment thereof comprising at least one component of the cellular MRN complex (i.e. Mre11, Rad50, Nbs1).

Exemplary eukaryotic polypeptides include, but are not limited to, Single-stranded DNA-binding protein (mitochondrial), Nuclear cap-binding protein subunit 1, Heat shock protein HSP 90-beta, Putative heat shock protein HSP 90-beta-3, Heat shock protein HSP 90-alpha, Transmembrane protein 263, ATP synthase subunit gamma (mitochondrial), Mitochondrial 2-oxoglutarate/malate carrier protein, Complement component 1 Q subcomponent-binding protein (mitochondrial), Mitochondrial import receptor subunit TOM22 homolog, Serine/threonine-protein phosphatase PGAM5 (mitochondrial), Voltage-dependent anion-selective channel protein 2, Histone H1.3, Protein WWC2, Transmembrane protein 33, HIG1 domain family member 1A (mitochondrial), CDK5 regulatory subunit-associated protein 2, Eukaryotic translation elongation factor 1 epsilon-1, DNA repair protein RAD50, Sideroflexin-4, Importin subunit alpha-4, E3 ubiquitin-protein ligase RBX1;E3 ubiquitin-protein ligase RBX1 (N-terminally processed), DNA-binding protein RFX7, ATP synthase subunit alpha, mitochondrial, Vimentin, Trafficking protein particle complex subunit 8, Pyruvate kinase PKM, GTP-binding nuclear protein Ran, Prohibitin-2, Importin subunit alpha-1, Synapsin-3, Peroxisome biogenesis factor 1, Nibrin, DnaJ homolog subfamily B member 6, DnaJ homolog subfamily B member 3, DnaJ homolog subfamily B member 8, DnaJ homolog subfamily B member 2, Cystatin-A;Cystatin-A, N-terminally processed, T-complex protein 1 subunit alpha, E3 ubiquitin-protein ligase TRIM21, Elongation factor 1-gamma, Double-strand break repair protein MRE11A, Heat shock protein 75 kDa (mitochondrial), Probable C-mannosyltransferase DPY19L1, Biogenesis of lysosome-related organelles complex 1 subunit 2, Nuclear pore complex protein Nup93, Leucine-rich repeat neuronal protein 4, Very-long-chain enoyl-CoA reductase, Peroxisomal sarcosine oxidase, Mitochondrial dicarboxylate carrier, Wings apart-like protein homolog, Cofilin-1, Destrin, Cofilin-2, Tubulin alpha-1B chain, Tubulin alpha-1A chain, Tubulin alpha-1C chain, Tubulin alpha-4A chain, Tubulin alpha-3C/D chain, Tubulin alpha-3E chain, Heat shock 70 kDa protein 1B, Heat shock 70 kDa protein 1A, Puromycin-sensitive aminopeptidase-like protein, Serine/arginine repetitive matrix protein 3, Protein PAT1 homolog 2, Centrosomal protein of 290 kDa, Zinc finger protein 25, ADAMTS-like protein 3, CAP-Gly domain-containing linker protein 4, EH domain-binding protein 1-like protein 1, Synaptotagmin-like protein 5, Guanine nucleotide exchange factor DBS, Nuclear transition protein 2, Protein bicaudal D homolog 1 and Putative helicase Mov10L1.

According to one embodiment, the nuclease and the second polypeptide are translationally fused, e.g. a fusion protein.

As used herein, the term “fused” refers to a protein or peptide which is physically associated with another protein or peptide, which naturally do not form a complex. In some embodiments, fusion is typically by a covalent linkage, however, other types of linkages are encompassed in the term “fused” include, for example, linkage via an electrostatic interaction, or a hydrophobic interaction and the like. Covalent linkage can encompass linkage as a fusion protein or chemically coupled linkage, for example via a disulfide bound formed between two cysteine residues.

According to a specific embodiment the fused molecule is a “fusion polypeptide” or “fusion protein”, a protein created by joining two or more heterologously related polypeptide sequences together. The fusion polypeptides encompassed in this invention include translation products of a chimeric nucleic acid construct that joins the DNA sequence encoding a DNA editing agent with the DNA sequence encoding a polypeptide capable of increasing HR to form a single open-reading frame. In other words, a “fusion polypeptide” or “fusion protein” is a recombinant protein of two or more proteins which are joined by a peptide bond.

The terms “fusion protein”, “chimera”, “chimeric molecule”, or “chimeric protein” are used interchangeably.

According to a specific embodiment, the nuclease comprises a Cas9-UL12 fusion protein (e.g. a fusion protein comprising Cas9 and amino acids 1-126 of an N-terminal fragment of UL12). Exemplary amino acid sequences of such fusion proteins are set forth in SEQ ID NOs: 27 and 28.

According to one embodiment, the DNA editing agent having double strand DNA cutting activity (the nuclease) and the polypeptide capable of increasing homologous recombination are joined or linked or fused, using recombinant techniques, at the amino-terminus or carboxyl-terminus.

As mentioned, the gene of the genome cell is mutated such that it is converted into a selection marker. In one embodiment, the mutation is a single point mutation. In another embodiment, the mutation comprises no more than two point mutations and preferably no more than three point mutations.

According to a particular embodiment, the gene of the genome of the cell is converted into a selection marker using the CRISPR system.

The selection marker may be a positive or a negative selection marker.

In one embodiment, the selection marker is a protein.

In another embodiment, the selection marker is an RNA (e.g. miRNA).

Preferably, the selection marker of this aspect of the present invention is a negative selection marker.

The phrase “negative selection marker” refers to a gene product (e.g. protein or RNA) that prevents the growth of a cell on selective medium (or under particular conditions) that carry the marker gene, but not of cells that do not carry the marker gene. Selection of cells that grow on the medium or under the particular conditions provides for the identification of cells that have eliminated or evicted the selectable marker genes. Exemplary conditions which the cell can be engineered to not withstand include a temperature, an osmotic stress, an oxidative stress, presence of a metabolite, absence of a metabolite, a pH or a density of cells.

Any endogenous gene of the genome can be converted into a selection marker as long as mutation thereof does not affect the function or viability of the cell and so long as it has a sequence which, when mutated, encodes the selectable marker.

According to a particular embodiment, the endogenous gene is a housekeeping gene.

Exemplary genes which may be mutated in human cells include Transcription initiation factor TFIID subunit 1 (TAF1) or E1 ubiquitin-activating enzyme (see for example Salvat et al, European Journal of Biochemistry, Volume 267, Issue 12, 2000, pages 3712-3722, the contents of which are incorporated herein by reference). An exemplary sequence of TAF1 is set forth in SEQ ID NO: 29, which is encoded by the nucleic acid sequence as set forth in SEQ ID NO: 30.

The present inventors have shown that insertion of a G176D mutation on TAF1, renders the encoded protein a negative selectable marker which prevents the growth of cells at a temperature of about 39.5° C.

As mentioned herein above, for introduction of the mutation via genome editing (e.g. CRISPR editing), a donor repair is required which contains the desired sequence.

According to one embodiment, the donor repair template is an RNA oligonucleotide.

According to one embodiment, the donor repair template is a DNA oligonucleotide.

According to one embodiment, the donor repair template is a single-stranded donor oligonucleotides (ssODN).

According to one embodiment, the donor repair template is a double-stranded donor oligonucleotide (dsODN).

In some embodiments, the present invention provides a recombinant donor repair template comprising two homology arms that are homologous to portions of a target DNA sequence (e.g., target gene or locus) at either side of a Cas nuclease (e.g., Cas9 nuclease) cleavage site. In certain instances, the recombinant donor repair template comprises two homology arms that flank the reporter cassette and are homologous to portions of the target DNA at either side of the Cas nuclease cleavage site.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb.

The donor repair template can be cloned into an expression vector. Conventional viral and non-viral based expression vectors known to those of ordinary skill in the art can be used.

In place of a recombinant donor repair template, a single-stranded oligodeoxynucleotide (ssODN) donor template can be used for homologous recombination-mediated repair. An ssODN is useful for introducing short modifications within a target DNA. For instance, ssODN are suited for introducing point mutations. ssODNs can contain two flanking, homologous sequences on each side of the target site of Cas nuclease cleavage and can be oriented in the sense or antisense direction relative to the target DNA. Each flanking sequence can be at least about 10 base pairs (bp), e.g., at least about 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1 kb, 2 kb, 4 kb, or longer. In some embodiments, each homology arm is about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about 1 kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about 1 kb, about 500 bp to about 2 kb, about 500 bp to about 4 kb, about 1 kb to about 2 kb, about 1 kb to about 2 kb, about 1 kb to about 4 kb, or about 2 kb to about 4 kb. The ssODN can be at least about 25 nucleotides (nt) in length, e.g., at least about 25 nt, 30 nt, 35 nt, 40 nt, 45 nt, 50 nt, 55 nt, 60 nt, 65 nt, 70 nt, 75 nt, 80 nt, 85 nt, 90 nt, 95 nt, 100 nt, 150 nt, 200 nt, 250 nt, 300 nt, or longer. In some embodiments, the ssODN is about 25 to about 50; about 50 to about 100; about 100 to about 150; about 150 to about 200; about 200 to about 250; about 250 to about 300; or about 25 nt to about 300 nt in length.

In some embodiments, the ssODN template comprises at least one, e.g., 1, 2, 3, 4, 5, 6, 7, 8, 9, 10, 20, 30, 40, 50, 60, 70, 80, 90, 100, 200, 300, 400, 500, or more modified nucleotides described herein. In some instances, at least 1%, 5%, 10%, 20%, 30%, 40%, 50%, 60%, 70%, 80%, 90%, or 99% of the sequence of the ssODN includes a modified nucleotide. In some embodiments, the modified nucleotides are located at one or both of the terminal ends of the ssODN. The modified nucleotides can be at the first, second, third, fourth, fifth, sixth, seventh, eighth, ninth, or tenth terminal nucleotide, or any combination thereof. For instance, the modified nucleotides can be at the three terminal nucleotides at both ends of the ssODN template. Additionally, the modified nucleotides can be located internal to the terminal ends.

Once mutated cells are obtained which carry an endogenous selection marker (e.g. a negative selection marker), they may be co-transfected with:
(i) a first DNA editing agent for specifically disrupting selection marker activity of the selection marker; and
(ii) a second DNA editing agent for specifically editing the genome at the target sequence of interest.

The term “co-transfecting” according to this aspect of the present invention refers to the simultaneous (i.e. concomitant) transfection of both the first and the second DNA editing agent. Thus, in one embodiment, the first and second DNA editing agent are provided as two distinct agents (e.g. on separate expression vectors) and the two agents are introduced into the cell simultaneously. In another embodiment, the first and second DNA editing are provided as one agent (e.g. encoded on a single expression vector) and only one vector is introduced into the cell.

The first and/or second DNA editing agent typically comprise a nuclease—examples of which include meganuclease (MN), a zinc finger nuclease (ZFN), a transcription activator-like effector nuclease (TALEN) and Cas9, as further described herein above.

Preferably, the first and the second DNA editing agents are of the same type—use the same nuclease. When the first and the second DNA editing agents are both Cas9, the first DNA editing agent further comprises at least a gRNA which specifically targets the selection marker gene and the second DNA editing agent comprises at least a gRNA which specifically targets the target sequence of interest.

The first DNA editing agent typically also comprises a recombinant donor repair template which comprises the non-mutated sequence of the endogenous gene. In one embodiment, the donor repair template comprises the wild-type sequence of the endogenous gene (i.e. the correct non-mutated sequence).

The second DNA editing agent typically may also comprise a recombinant donor repair template or a single-stranded oligodeoxynucleotide (ssODN) donor template which is introduced into the target sequence of interest.

Following transfection, the cells are cultured under conditions that enrich for cells that do not comprise the selection marker. The cells are cultured for at least one day, two days, three days, four days, five days, six days or at least one week.

Thus, for example if the selection marker renders the cells sensitive to high temperatures (e.g. cells cannot survive at a temperature of 39.5° C.), upon disruption of the marker activity, cells that survive high temperatures are devoid of the selection marker. The present inventors have shown that culturing under conditions that enrich for cells that do not comprise the selection marker inherently enriches for cells that have undergone the genome editing event at the target sequence of interest.

In order to confirm a cell has undergone the required genome editing event, the sequence of the genome of the cells may be analyzed.

Methods for detecting sequence alteration are well known in the art and include, but not limited to, DNA sequencing (e.g., next generation sequencing), electrophoresis, an enzyme-based mismatch detection assay and a hybridization assay such as PCR, RT-PCR, RNase protection, in-situ hybridization, primer extension, Southern blot, Northern Blot and dot blot analysis. Various methods used for detection of single nucleotide polymorphisms (SNPs) can also be used, such as PCR based T7 endonuclease, Hetroduplex and Sanger sequencing, or PCR followed by restriction digest to detect appearance or disappearance of unique restriction site/s.

In some embodiments, the cells obtained following the method disclosed herein are scarless i.e. the genome of the cell is wild-type (apart from the genome editing event at the target sequence of interest). No remnant of the selectable marker remains.

The present inventors contemplate additional methods for selecting genome-edited cells, wherein the cells are not left completely scarless, but a remnant remains of the selectable marker.

Thus, according to another aspect of the present invention there is provided a method of selecting a cell which harbors a genome-editing event at a target sequence of interest comprising:
(a) co-transfecting cells having a genome with:
   (i) a first DNA editing agent for specifically introducing a mutation on a first gene of said genome, said mutation rendering said first gene a selection marker; and
   (ii) a second DNA editing agent for specifically editing the genome at the target sequence of interest; and
(c) culturing the cells under conditions that enrich for cells that comprise said selection marker, thereby selecting a cell which harbors the genome-editing event.

The method of this aspect of the present invention is similar to the one described herein above, except for this method, the first DNA editing agent is for converting an endogenous gene of the genome of the cell into a selection marker, whereas in the method described herein above, the first DNA editing agent is for reverting the selection marker gene into its wild-type.

Types of cells which can be genome edited according to this aspect of the present invention are described herein above, except that they don't need to be pre-edited (as detailed herein above).

Thus, the first DNA editing agent is responsible for editing an endogenous gene of the genome of the cell such that it is converted into a selection marker.

Selection markers and endogenous genes which can be converted include those described herein above.

Typically, the selection marker of this aspect of the present invention is a positive selection marker.

A “positive selection marker” as used herein, refers to a gene product (e.g. protein or RNA) that allows growth on selective medium (or under particular conditions) of cells that carry the marker gene, but not of cells that do not carry the marker gene. Selection is for cells that grow on the selective medium or under said conditions (showing acquisition of the marker) and is used to identify transformants. Exemplary conditions which the cell can be engineered to withstand include a temperature, presence or absence of a metabolite (see for example Ayusawa et al., Somatic Cell Genetics, 1981, Sep. 7(5) pages 523-534; Patel et al., 2003, The Journal of Biological Chemistry 278, pages 19436-19441; Altboum et al, Journal of Bacteriology, 1990, pages 3898-3904; and Chu et al., 1972, PNAS, Vol. 69, No. 11, pages 3459-3463) a pH or a density of cells.

In one embodiment, the conditions which the cell can be engineered to withstand is the presence of a toxic chemical, examples of which include, but are not limited to cycloheximide (CHX), hydroxyurea (HU), Methotrexate, proteasome inhibitors, alpha-amanitine, 8-azaguanine and DNA damaging agents such as Bortezomib, Carfilzomib, Ixazomib, Marizomib, Oprozomib, Delanzomib.

According to a particular embodiment, the endogenous gene is a housekeeping gene.

Exemplary genes which may be mutated in human cells include but are not limited to Ribosomal Protein L36a (RPL36A), dihydrofolate reductase (DHFR), RNA polymerase (SEQ ID NO: 35) target of alpha amanitine), ribonucleotide reductase (RNR), DNA polymerase and a proteasome subunit.

Insertion of a P54Q mutation on RPL36A (amino acid sequence as set forth in SEQ ID NO: 31, nucleic acid sequence SEQ ID NO: 32) renders the encoded protein a positive selectable marker which allows the growth of cells in the presence of cycloheximide.

Insertion of a L22Y mutation on DHFR (amino acid sequence as set forth in SEQ ID NO: 33) renders the encoded protein a positive selectable marker which allows the growth of cells in the presence of methotrexate.

Insertion of a S188T mutation on RNR (amino acid sequence as set forth in SEQ ID NO: 34) renders the encoded protein a positive selectable marker which allows the growth of cells in the presence of hydroxyurea.

Insertion of a mutation on Thymidylate synthase renders the encoded protein a positive selectable marker which allows the growth of cells in the presence of methotrexate

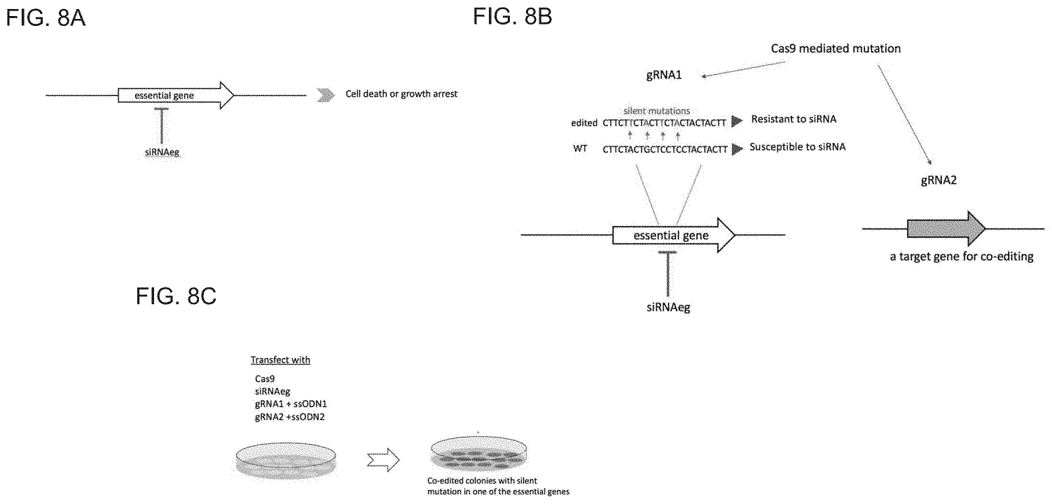

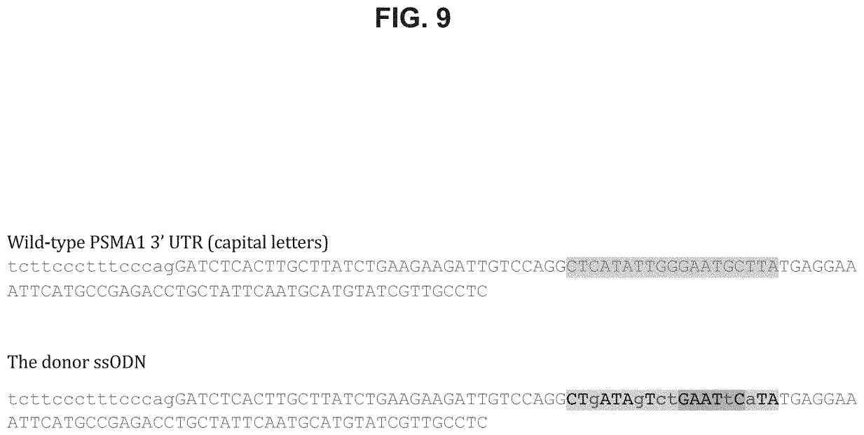

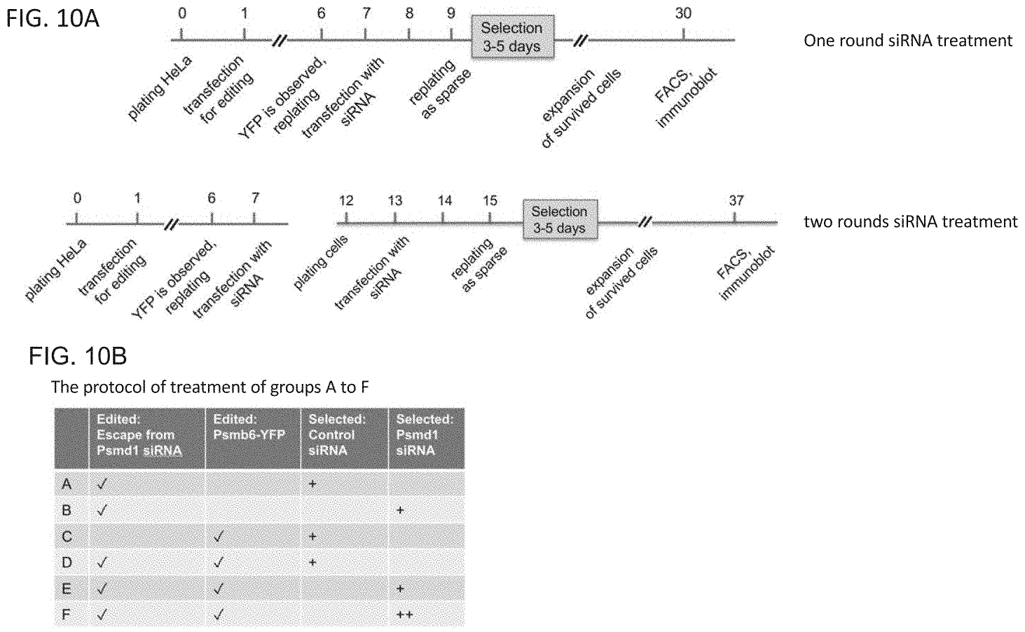

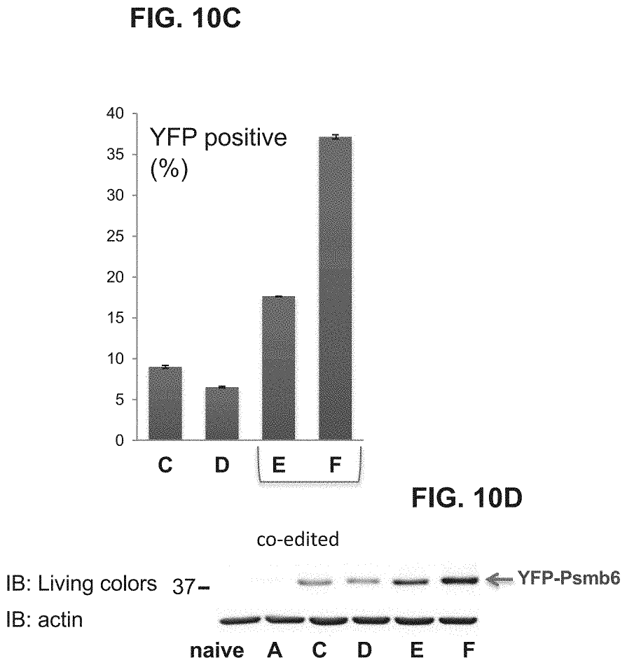

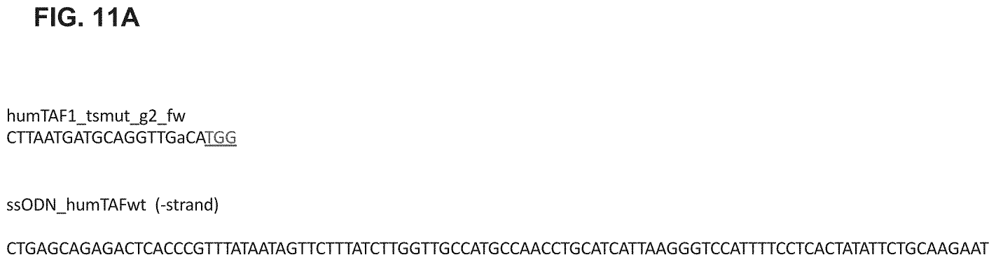

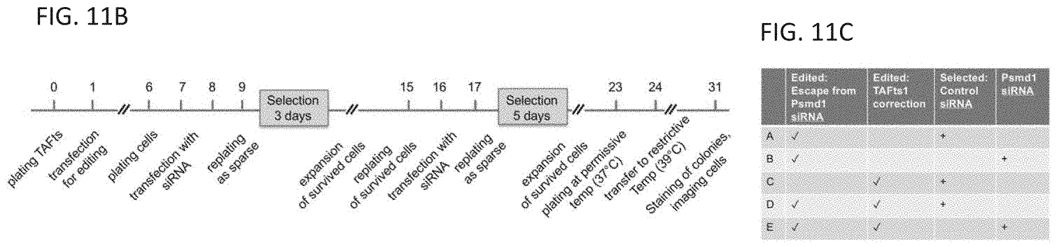

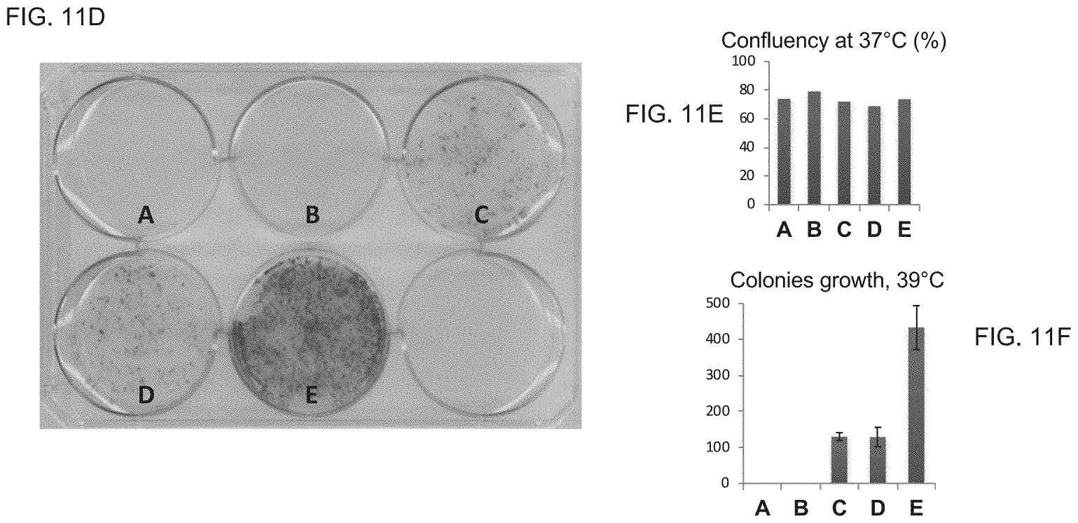

According to a specific embodiment, the endogenous gene is an essential gene, as illustrated in FIGS. 8A-B.

As used herein, the term “essential gene” refers to a gene which is necessary to for cell growth and/or survival.

Examples of essential genes include, but are not limited to DNA polymerases, RNA polymerase, genes encoding ribosome components, genes encoding proteasome components and genes encoding translation components.

For this embodiment, the first DNA editing agent specifically introduces at least one, at least two, at least three or more mutations on an essential gene such that it is no longer a target for an RNA silencing agent—i.e. it is not capable of hybridizing to the RNA silencing agent, as the RNA silencing agent is directed towards the wild-type gene. The transfection step will also include the RNA silencing agent.

As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

The second DNA editing agent is for editing the target of interest (as described herein above).

The first and/or second DNA editing agent of this aspect of the present invention typically comprise a nuclease—examples of which include meganuclease (MN), a zinc finger nuclease (ZFN), a transcription activator-like effector nuclease (TALEN) and Cas9, as further described herein above.

Preferably, the first and the second DNA editing agents are of the same type—use the same nuclease. When the first and the second DNA editing agents are both Cas9, the first DNA editing agent further comprises at least a gRNA which specifically targets the endogenous gene (such that it can be mutated and converted to a selectable marker) and the second DNA editing agent comprises at least a gRNA which specifically targets the target sequence of interest.

The first DNA editing agent typically also comprises a recombinant donor repair template or a single-stranded oligodeoxynucleotide (ssODN) donor template which comprises the mutated sequence of the endogenous gene. In one embodiment, the recombinant donor repair template or single-stranded oligodeoxynucleotide (ssODN) donor template comprises a single point mutation of the endogenous gene. In another embodiment, the recombinant donor repair template or single-stranded oligodeoxynucleotide (ssODN) donor template comprises at least two mutations, and preferably no more than three mutations of the endogenous gene.

The second DNA editing agent typically may also comprises a recombinant donor repair template or a single-stranded oligodeoxynucleotide (ssODN) donor template which is introduced into the target sequence of interest.

Following transfection, the cells are cultured under conditions that enrich for cells that comprise the selection marker.

Thus, for example if the selection marker renders the cells resistant to a particular agent (e.g. cycloheximide), upon creation of the marker activity, cells that survive in the presence of the agent have been successfully genome-edited to comprise the selection marker. The present inventors have shown that culturing under conditions that enrich for cells that comprise the selection marker inherently enriches for cells that have undergone the genome editing event at the target sequence of interest.

According to yet another aspect of the present invention, there is provided a method of selecting a cell which harbors a genome-editing event at a target sequence of interest comprising:
(a) co-transfecting cells having a genome with:
   (i) a first DNA editing agent for introducing a first mutation into a first gene of a genome of said cells, said first mutation renders said first gene a first selection marker having a selection marker activity which imparts susceptibility of said cells to a condition;
   (ii) a second DNA editing agent for introducing a second mutation into a second gene of a genome of said cells, said second mutation renders said second gene a second selection marker having a selection marker activity which imparts resistance of said cells to an agent; and
   (iii) a third editing agent for editing the genome at the target sequence of interest;
(b) culturing the cells in the presence of said agent so as to enrich for cells that comprise said second selection marker;
(c) co-transfecting said cells that comprise said second selection marker with:
   (i) a fourth DNA editing agent for disrupting said selection marker activity of said first selection marker;
   (ii) a fifth DNA editing agent for disrupting said selection marker activity of said second selection marker; and
(d) culturing said cells under conditions that enrich for cells that do not comprise said first selection marker, thereby selecting a cell harboring the genome-editing event.

This aspect of the present invention is a combination of the two aspects described herein above.

The starting cells of this aspect are not “pre-edited” such that any cell population is envisaged (as described herein above).

This method requires two co-transfection and selection steps. The first co-transfection allows for co-editing of the genome at three distinct sites:
(i) a first site—rendering a first endogenous gene a first selection marker having a selection marker activity which imparts susceptibility of said cells to a condition;
(ii) a second site—rendering a second endogenous gene a second selection marker having a selection marker activity which imparts resistance of cells to an agent; and
(iii) a third site—the target sequence of interest which is edited.

The cells are initially cultured in the presence of an agent so as to enrich for cells that comprise the second selection marker.

Once the cells are selected, a second co-transfection step is carried out to reverse the selection marker activity created in the first step.

The cells are then cultured under conditions that enrich for cells that do not comprise the first selection marker (e.g. cultured at temperatures between 39-40° C. such as 39.5° C.), thereby selecting a cell harboring the genome-editing event.

The DNA editing agents of some embodiments of the invention may be introduced into target cells (e.g. eukaryotic cells) using DNA delivery methods (e.g. by expression vectors) or using DNA-free methods.

According to one embodiment, the DNA editing agents can be provided as RNA to the cell.

Thus, it will be appreciated that the present techniques relate to introducing the DNA editing agents using DNA-free methods such as RNA transfection (e.g. mRNA transfection), or Ribonucleoprotein (RNP) transfection (e.g. protein-RNA complex transfection, e.g. Cas9-sgRNA complex).

According to a specific embodiment, the DNA editing agents (e.g. comprising, for example, Cas9 and sgRNA) are provided using DNA delivery methods (e.g. via plasmid).

According to a specific embodiment, the DNA editing agents (e.g. comprising, for example, Cas9 and sgRNA) are provided using DNA-free delivery methods (e.g. via RNA transfection).

According to a specific embodiment, the DNA editing agent (e.g. comprising, for example, Cas9) are provided as polypeptides.

According to a specific embodiment, the DNA editing agents (e.g. comprising, for example, Cas9 and sgRNA) are provided as protein-RNA complex transfection.

According to one embodiment, for expression of DNA editing agents of the invention in mammalian cells, a nucleic acid sequence encoding the DNA editing agent is inserted into at least one nucleic acid construct suitable for mammalian cell expression. Such a nucleic acid construct includes a promoter sequence for directing transcription of the nucleotide sequences in the target cell in a constitutive or inducible manner.

The nucleic acid construct (also referred to herein as an “expression vector”) of some embodiments of the invention includes additional sequences which render this vector suitable for replication and integration in eukaryotes (e.g., shuttle vectors). In addition, typical cloning vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

Eukaryotic promoters typically contain two types of recognition sequences, the TATA box and upstream promoter elements. The TATA box, located 25-30 base pairs upstream of the transcription initiation site, is thought to be involved in directing RNA polymerase to begin RNA synthesis. The other upstream promoter elements determine the rate at which transcription is initiated.

Preferably, the promoter utilized by the nucleic acid construct of some embodiments of the invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166).

Enhancer elements can stimulate transcription up to 1,000 fold from linked homologous or heterologous promoters. Enhancers are active when placed downstream or upstream from the transcription initiation site. Many enhancer elements derived from viruses have a broad host range and are active in a variety of tissues. For example, the SV40 early gene enhancer is suitable for many cell types. Other enhancer/promoter combinations that are suitable for some embodiments of the invention include those derived from polyoma virus, human or murine cytomegalovirus (CMV), the long term repeat from various retroviruses such as murine leukemia virus, murine or Rous sarcoma virus and HIV. See, Enhancers and Eukaryotic Expression, Cold Spring Harbor Press, Cold Spring Harbor, N.Y. 1983, which is incorporated herein by reference.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Polyadenylation sequences can also be added to the expression vector in order to increase the efficiency of mRNA translation. Two distinct sequence elements are required for accurate and efficient polyadenylation: GU or U rich sequences located downstream from the polyadenylation site and a highly conserved sequence of six nucleotides, AAUAAA, located 11-30 nucleotides upstream. Termination and polyadenylation signals that are suitable for some embodiments of the invention include those derived from SV40.

In addition to the elements already described, the expression vector of some embodiments of the invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

It will be appreciated that the individual elements comprised in the expression vector can be arranged in a variety of configurations. For example, enhancer elements, promoters and the like, and even the polynucleotide sequence(s) encoding the DNA editing agent and/or the polypeptide capable of increasing homologous recombination in a target cell can be arranged in a “head-to-tail” configuration, may be present as an inverted complement, or in a complementary configuration, as an anti-parallel strand. While such variety of configuration is more likely to occur with non-coding elements of the expression vector, alternative configurations of the coding sequence within the expression vector are also envisioned.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Thus, the type of vector used by some embodiments of the invention will depend on the cell type transformed. The ability to select suitable vectors according to the cell type transformed is well within the capabilities of the ordinary skilled artisan and as such no general description of selection consideration is provided herein. For example, bone marrow cells can be targeted using the human T cell leukemia virus type I (HTLV-I) and kidney cells may be targeted using the heterologous promoter present in the baculovirus Autographa californica nucleopolyhedrovirus (AcMNPV) as described in Liang C Y et al., 2004 (Arch Virol. 149: 51-60).

Recombinant viral vectors are useful for in vivo expression of recombinant systems since they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This contrasts with vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Various methods can be used to introduce the expression vector of some embodiments of the invention into eukaryotic cells (e.g. stem cells). Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of some embodiments of the invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Other than containing the necessary elements for the transcription and translation of the inserted coding sequence, the expression construct of some embodiments of the invention can also include sequences engineered to enhance stability, production, purification, yield or toxicity of the expressed peptide.

In cases where plant expression vectors are used, the expression of the coding sequence can be driven by a number of promoters. For example, viral promoters such as the 35S RNA and 19S RNA promoters of CaMV [Brisson et al. (1984) Nature 310:511-514], or the coat protein promoter to TMV [Takamatsu et al. (1987) EMBO J. 6:307-311] can be used. Alternatively, plant promoters such as the small subunit of RUBISCO [Coruzzi et al. (1984) EMBO J. 3:1671-1680 and Brogli et al., (1984) Science 224:838-843] or heat shock promoters, e.g., soybean hsp17.5-E or hsp17.3-B [Gurley et al. (1986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 1988, Methods for Plant Molecular Biology, Academic Press, NY, Section VIII, pp 421-463.

According to one embodiment, the expression vector comprises a nucleic acid sequence encoding a single DNA editing agent (e.g. nuclease and the DNA recognition unit).

According to one embodiment, the expression vector comprises nucleic acid sequences encoding a single nuclease and multiple DNA recognition units—i.e. gRNAs).

The agents described herein can be provided per se or as part of a kit for carrying out selection of a cell harboring a genome-editing event.

At its minimum, the kit comprises a DNA editing agent for specifically introducing a mutation into a gene, wherein said mutation converts an endogenous gene (having no selection marker activity) into a selection marker.

Alternatively or additionally, the kit may comprise a DNA editing agent for specifically introducing a mutation into a gene, wherein the mutation disrupts selection marker activity of the selection marker.

The DNA editing agent may be provided as DNA encoding the agent, RNA and/or as protein.

Examples of DNA editing agents are provided herein above. In one embodiment, the DNA editing agent comprises a DNA donor template and appropriate gRNAs. In another embodiment, the kit further comprises DNA encoding the DNA editing nuclease e.g. Cas9 nuclease. Specifically, the DNA editing agent may comprise a DNA agent encoding a Cas9 nuclease and appropriate gRNA together with the DNA donor template. Each of the agents may be encoded on a separate nucleic acid construct, on a single nucleic acid construct or as other combinations.

Additional agents that can be provided in the kit include the selection marker itself (as further described herein above) and or cells that have been pre-transformed so that endogenous genes thereof have been converted to selection markers.

The containers of the kits will generally include at least one vial, test tube, flask, bottle, syringe or other containers, into which a component may be placed, and preferably, suitably aliquoted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 1 is expressed in a DNA sequence format (e.g., reciting T for thymine), but it can refer to either a DNA sequence that corresponds to an Cas9 nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### GENERAL MATERIALS AND EXPERIMENTAL PROCEDURES

Cells and Cell Culture: Human embryonic kidney cells HEK293 were grown at 37° C. in a humidified incubator with 5.6% CO₂ in Dulbecco's modified Eagle's medium (DMEM; GIBCO, Life Technologies, Thermo Scientific, Waltham, MA) supplemented with 8% fetal bovine serum (GIBCO), 100 units/ml penicillin, and 100 μg/ml streptomycin. The restrictive temperature used for the HEK293 TAF1ts cells was 39.5° C. Light microscopy photography of cells was performed using an Olympus (Tokyo, Japan) IX70 microscope connected to a DVC camera.

Plasmids and transfection: The SpCas9/sgRNA expression plasmids were based on pX330-U6-Chimeric_BB-CBh-hSpCas9, (Addgene plasmid #42230; http://n2t.net/addgene:42230; RRID:Addgene_42230) (2), and Addgene plasmid #64324 (pU6-(BbsI)_CBh-Cas9-T2A-mCherry was a gift from Ralf Kuehn (Addgene plasmid #64324) (12). Guide RNA and ssODN sequences, as well as other primers used for PCR are listed in Table 1. Plasmid donor DNA constructs used pBlueScript KS—as a backbone. The homology arm DNA was amplified by PCR from the cell lines' genomic DNA and cloned into the backbone using the restriction sites noted in Table 1. The sequence for YFP was amplified from pSYFP2-C1 (pSYFP2-C1—Addgene plasmid #22878)(13). Transfections were performed by the calcium phosphate method as described (14), JetPEI® (Polyplus-transfection SA, Illkirch, France), or with polyethylenimine (PEI) 25K (Polysciences) prepared at 1 mg/ml and used similarly to the commercial JetPEI reagent.

Immunoblot and coimmunoprecipitation: Immunoblots were performed as previously described (14) using RIPA buffer (50 mM Tris-HCl pH 7.5, 150 mM NaCl, 1% Nonidet P-40 (v/v), 0.5% deoxycholate (v/v), 0.1% SDS (w/v)) supplemented with cocktails of protease inhibitors and serine/threonine and tyrosine phosphatase inhibitors (Apex Bio). Antibodies used were: anti-β-actin (Sigma, St. Louis, MO), and the polyclonal Living Colors antibody (Clontech), to detect SYFP. Horseradish peroxidase-conjugated secondary antibodies were from Jackson ImmunoResearch Laboratories, West Grove, PA. Enhanced chemiluminescence was performed with the EZ-ECL kit (Biological Industries, Kibbutz Beit Haemek, Israel) and signals were detected by the ImageQuant LAS 4000 (GE Healthcare, Piscataway, NJ).

Cells and Cell Culture: Human embryonic kidney cells HEK293 were grown at 37° C. in a humidified incubator with 5.6% CO₂ in Dulbecco's modified Eagle's medium (DMEM; GIBCO, Life Technologies, Thermo Scientific, Waltham, MA) supplemented with 8% fetal bovine serum (GIBCO), 100 units/ml penicillin, and 100 μg/ml streptomycin. The restrictive temperature used for the HEK293 TAF1ts cells was 39.5° C. Light microscopy photographs of cells were performed using an Olympus (Tokyo, Japan) IX70 microscope connected to a DVC camera.

Plasmids and transfection: The SpCas9/sgRNA expression plasmids were based on pX330-U6-Chimeric_BB-CBh-hSpCas9, a gift from Feng Zhang (Addgene plasmid #42230; http://n2t.net/addgene:42230; RRID:Addgene_42230) (2), and Addgene plasmid #64324 (pU6-(BbsI)_CBh-Cas9-T2A-mCherry was a gift from Ralf Kuehn (Addgene plasmid #64324) (12). Guide RNA and ssODN sequences, as well as other primers used for PCR are listed in Table I. Plasmid donor DNA constructs used pBlueScript KS—as a backbone. The homology arm DNA was amplified by PCR from the cell lines' genomic DNA and cloned into the backbone using the restriction sites noted in Table I. The sequence for YFP was amplified from pSYFP2-C1 (pSYFP2-C1 was a gift from Dorus Gadella (Addgene plasmid #22878)(13). Transfections were done by the calcium phosphate method as described (14), JetPEI® (Polyplus-transfection SA, Illkirch, France), or with polyethylenimine (PEI) 25K (Polysciences) prepared at 1 mg/ml and used similarly to the commercial JetPEI reagent.

Immunoblot and co-immunoprecipitation: Immunoblots were performed as previously described (14)using RIPA buffer (50 mM Tris-HCl pH 7.5, 150 mM NaCl, 1% Nonidet P-40 (v/v), 0.5% deoxycholate (v/v), 0.1% SDS (w/v)) supplemented with cocktails of protease inhibitors and serine/threonine and tyrosine phosphatase inhibitors (Apex Bio). Antibodies used were: anti-β-actin (Sigma, St. Louis, MO), and the polyclonal Living Colors antibody (Clontech), to detect SYFP. Horseradish peroxidase-conjugated secondary antibodies were from Jackson ImmunoResearch Laboratories, West Grove, PA. Enhanced chemiluminescence was performed with the EZ-ECL kit (Biological Industries, Kibbutz Beit Haemek, Israel) and signals were detected by the ImageQuant LAS 4000 (GE Healthcare, Piscataway, NJ).

TABLE 1
Guide for C-terminus of human PSMB6

| | |
|---|---|
| CR_B6_stop_g2_fw | caccgTAGAATCCCAGGATTCAGGC (SEQ ID NO: 1) |
| CR_B6_stop_g2_re | aaacGCCTGAATCCTGGGATTCTAc (SEQ ID NO: 2) |

Primers to make human PSMB6-YFP donor template in pBluescript KS-with 1
kb homology arms

| | |
|---|---|
| SalI_b6_frg1_fw | Ctcgaggtcgaccactattctgccatcctgcaggtcctacateg (SEQ ID |
| | NO: 3) |
| HindIII_b6_frg1_re | Ggtggcaagcttggcgggtggtaaagtggcaacggcgaatttggg (SEQ |
| | ID NO: 4) |
| HindIII_ATG_Clover/ | Cccgccaagcttgccaccatggtgagcaagggcgagg (SEQ ID NO: |
| YFP_fw | 5) |
| BamHI_Clover/YFP_rev | Gattcaggatccagctcgagatctgagtccggacttgtacagctcg (SEQ ID |
| | NO: 6) |
| BamHI_b6_Frg2_fw | Cgagctggatcctgaatcctgggattctagtatgcaataagagatg (SEQ ID |
| | NO: 7) |
| XbaI_b6_Frg2_re | Ggccgctctagagcagtgagccaagaccaggctactgcactccagc (SEQ |
| | ID NO: 8) |

Guide for targeting human TAF1, aa716

| | |
|---|---|
| TAF1_g1_fw | caccGGACCCTTAATGATGCAGGT |
| | (SEQ ID NO: 9) |
| TAF1_g1_re | aaacACCTGCATCATTAAGGGTCC |
| | (SEQ ID NO: 10) |

ssODN for creating ts mutation (G716D) in human TAF1
TCTGAGCAGAGACTCACCCGTTTATAATAGTTCTTTATCTTGGTTGCCATGt
CAACCTGCATCATTAAGGGTCCATTTTCCTCACTATATTCTGCAAGAATAA
(SEQ ID NO: 11)
Primers flanking the TAF1 guide site, to amplify 554 bp fragment

| | |
|---|---|
| TAF1_hum_gen554_fw | Gcagaacccatacatggatatggagg (SEQ ID |
| | NO: 12) |
| TAF1_hum_gen554_re | Tatggtatatgttcacagattaccag (SEQ ID NO: |
| | 13) |

Guide targeting the mutant human TAF1

| | |
|---|---|
| humTAF1_tsmut_g2_fw | caccgCTTAATGATGCAGGTTGaCA |
| | (SEQ ID NO: 14) |
| humTAF1_tsmut_g2_re | aaacTGtCAACCTGCATCATTAAGc |
| | (SEQ ID NO: 15) |

ssODN for correcting ts mutation to make wt human TAF1
CTGAGCAGAGACTCACCCGTTTATAATAGTTCTTTATCTTGGTTGCCATGC
CAACCTGCATCATTAAGGGTCCATTTTCCTCACTATATTCTGCAAGAAT
(SEQ ID NO: 16)
Control guide non-targeting in human

| | |
|---|---|
| BFP_g2_fw | caccgCTGCACGCCGTGGGTCAGGG |
| | (SEQ ID NO: 17) |
| BFP_g2_re | aaacCCCTGACCCACGGCGTGCAGc |
| | (SEQ ID NO: 18) |

Control ssODN
ACCGGCAAGCTGCCCGTGCCCTGGCCCACCCTCGTGACCACCCTGACATA
CGGCGTGCAGTGCTTCAGCCGCTACCCCGACCACATGAAGCAGCACGAC
(SEQ ID NO: 19)

### Results

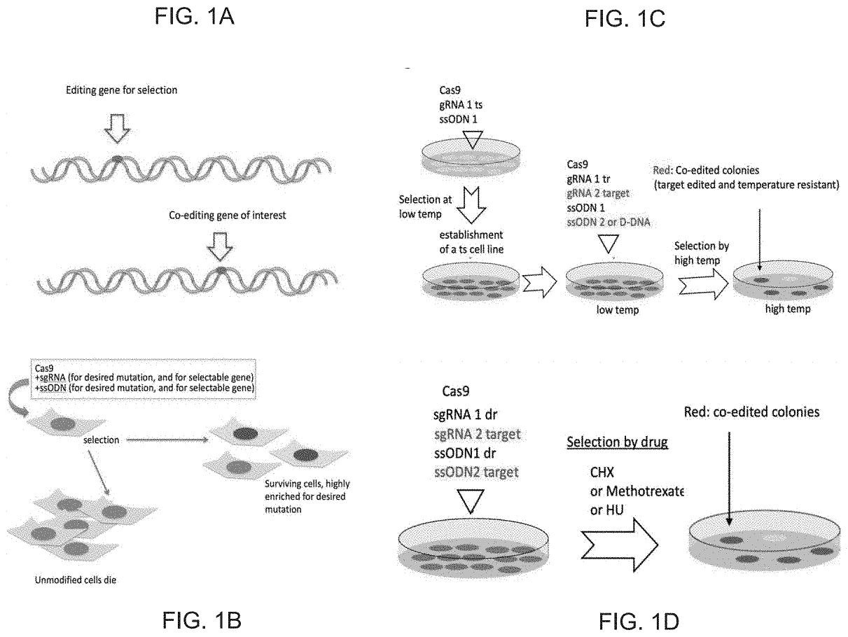

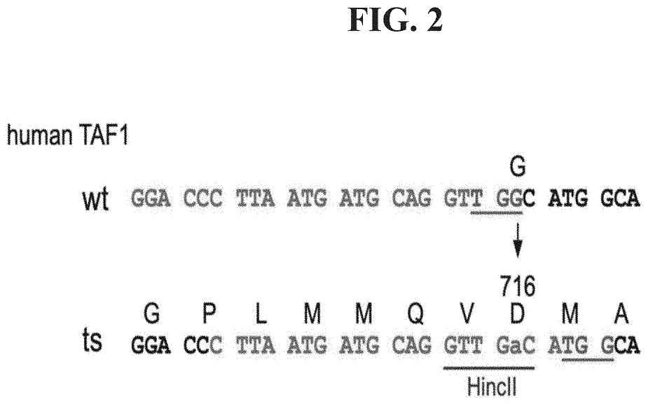

To achieve scarless DNA editing, the general strategy depicted in FIG. 1A was used. An endogenous gene was mutated (so as to serve as a selectable gene) and a gene of interest was simultaneously edited, a strategy referred to herein as co-editing. The rationale is that once the selectable gene is generated by Cas9 mediated HDR, the probability of the second concurrent hit of HDR is higher, and therefore a high incidence of gene editing is obtained (FIG. 1B). Furthermore, the process is such that the selection gives rise to colony formation and therefore overrides the need for laborious single cell culturing.

Establishment of the HEK293 ts Cell Line

A temperature-sensitive cell line, referred to herein as human embryonic kidney (HEK)293 TAFts was established. This cell line was engineered based on BHK21 ts13, a cell line bearing a well-defined point mutation, G690D, in the TAF_{II}-250 (TAF1) gene on the X chromosome, the largest component of the basal transcription complex TFIID (8). These cells grow at permissive temperature (37° C.), but die when incubated for several days at the restrictive temperature of 39.5° C. In human cells, the point mutation is TAF1 G716D, in exon 13 of the TAF1 gene on the X chromosome. HEK293 have three X chromosomes, and the clones that were isolated each had one allele with the ts mutation, and different insertions or deletions (indels) in the other two alleles (verified by sequencing (not shown)). The HEK293 TAF1ts cells grew at the permissive temperature of 37° C., but did not survive at the restrictive temperature (39.5° C.).

Temperature Sensitivity as a Selectable Marker

To test the co-editing protocol, the PSMB6 gene was edited to create a PSMB6-YFP fusion protein (FIG. 3A). HEK293 ts cells were transfected with the Cas9/guide plasmids targeting the PSMB6 site, along with the respective donor DNA, and with Cas9/guide and ssODN for correcting the ts mutation in TAF1. Unselected cells were grown at 37° C., while the selected cells were grown at 39.5° C. When non-specific sgRNA or ssODN were used, no HEK293 TAF1ts cell growth was observed at 39.5° C., suggesting spontaneous reversion is a very rare event if any (FIG. 3B). YFP was expressed in nearly 90% of the heat-selected colonies, indicating a very high rate of co-editing. To verify that the YFP expression was indeed from PSMB6-YFP, the colonies were harvested as a pool, and were analyzed by Western blotting (FIG. 3C), in comparison to the pools of unselected cells. The results show that the selected cells were greatly enriched for PSMB6-YFP. By loading dilutions of the selected cells' extract, it could be estimated that the enrichment achieved was in the range of 50-fold (FIG. 3C).

The TAF1ts system offered a selectable editing reaction that is “scarless”, that is, after editing, the gene used for selection is restored to the wt sequence. Furthermore, after incubation at 39.5° C., the colonies of CRISPR-edited cells can be easily picked and transferred to new plates, obviating the need for further single-cell cloning. In this manner, confirmed mutant cell lines can be obtained in as little as one month.

Cycloheximide Based Co-Editing Selection

In yeast, point mutations in ribosomal proteins enable the yeast to be resistant to cycloheximide, such as the P56Q mutation in L41 (9). Since the human and yeast proteins are highly homologous, sgRNA and ssODN were designed to target the human homolog, RPL36A, making the P54Q mutation (FIG. 4A). Using this strategy, naive HEK293 cells were co-edited with the Cas9/sgRNA plasmids for RPL36A and for PSMB6, along with the respective donor DNAs. As found with the ts cell co-editing a high percentage of PSMB6-YFP co-edited cells were obtained when the cells were selected with cycloheximide (FIG. 4C). The selected clones were isolated, and verified that they expressed the expected PSMB6-YFP protein (FIG. 4B), and also had the expected P54Q mutation (verified by sequencing). In the clones analyzed, one allele of RPL36A was mutated to P54Q, and the second allele remained wt.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A kit for selecting a cell harboring a genome-editing event at a target sequence of interest, comprising:
(i) a first DNA editing agent for specifically introducing a mutation into a first gene so as to generate a first selection marker which imparts resistance to a selection agent, wherein said target sequence of interest and said first gene are distinct; and
(ii) said selection agent; and/or
(iii) a second DNA editing agent for specifically introducing a mutation into said first gene, wherein said mutation disrupts selection marker activity of said first selection marker.

2. A kit for selecting a cell harboring a genome-editing event at a target sequence of interest, wherein a first gene of a genome of said transformed cell is mutated so as to render said first gene a first selection marker, wherein said kit comprises:
(i) a first DNA editing agent for specifically introducing a mutation into said first gene, wherein said mutation disrupts selection marker activity of said first selection marker, wherein said target sequence of interest and said first gene are distinct;
(ii) a second DNA editing agent for specifically introducing a mutation into said first gene so as to generate said first selection marker;
(iii) a third DNA editing agent for specifically introducing a mutation into a second gene of a genome of said transformed cell so as to render said second gene a second selection marker; and/or
(iv) a fourth DNA editing agent for specifically introducing a mutation into said second gene which disrupts marker activity of said second selection marker.

3. The kit of claim 1, comprising:
(i) a first DNA editing agent for specifically introducing a mutation into a first gene, said first gene being an essential gene, so as to generate a first selection marker which imparts resistance to a selection agent, wherein said target sequence of interest and said first gene are distinct; and
(ii) said selection agent.

4. The kit of claim 2, further comprising a selection agent, wherein said first selection marker or said second selection marker imparts resistance to said selection agent.

5. The kit of claim 3, wherein said selection agent is an RNA silencing agent.

6. The kit of claim 5, wherein said RNA silencing agent is an siRNA.

7. The kit of claim 1, wherein said first gene is a housekeeping gene or an essential gene.

8. The kit of claim 1, further comprising:
(iii) a third DNA editing agent for specifically introducing a mutation into a second gene of a genome of said transformed cell so as to render said second gene a second selection marker; and/or
(iv) a fourth DNA editing agent for specifically introducing a mutation into said second gene which disrupts marker activity of said second selection marker.

9. The kit of claim 2, wherein said first selection marker imparts sensitivity to a condition or resistance to an agent.

10. The kit of claim 8, wherein said first selection marker imparts sensitivity to a condition and said second selection marker imparts resistance to an agent or vice versa.

11. The kit of claim 9, wherein said condition is a temperature.

12-13. (canceled)

14. The kit of claim 1, wherein said first gene is an essential gene and said agent is an RNA silencing agent directed towards said essential gene.

15. The kit of claim 1, wherein said first DNA editing agent comprises a nuclease selected from the group consisting of a meganuclease (MN), a zinc finger nuclease (ZFN), a transcription activator-like effector nuclease (TALEN) and a clustered regularly interspaced short palindromic repeat (CRISPR)-associated nuclease (Cas9).

16. The kit of claim 1, wherein said second DNA editing agent comprises a nuclease selected from the group consisting of a meganuclease (MN), a zinc finger nuclease (ZFN), a transcription activator-like effector nuclease (TALEN) and a clustered regularly interspaced short palindromic repeat (CRISPR)-associated nuclease (Cas9).

17. The kit of claim 1, wherein said first and/or said second DNA editing agent comprise Cas9 and a guide RNA (gRNA).

18. The kit of claim 2, wherein said first DNA editing agent further comprises a first DNA donor template comprising a nucleic acid sequence which encodes a wild-type sequence of said first gene.

19. The kit of claim 2, wherein said second DNA editing agent further comprises a second DNA donor template comprising a nucleic acid sequence which encodes a mutated sequence of said first gene.

20. The kit of claim 1, wherein said first DNA editing agent further comprises a first DNA donor template comprising a nucleic acid sequence which encodes a mutated sequence of said first gene and wherein said second DNA editing agent further comprises a second DNA donor template comprising a nucleic acid sequence which encodes a wild-type sequence of said first gene.

21-22. (canceled)

23. A method of selecting a cell which harbors a genome-editing event at a target sequence of interest comprising:
(a) co-transfecting cells having a genome with:
(i) a first DNA editing agent for specifically introducing a mutation on a first gene of said genome, said mutation rendering said first gene a selection marker which imparts resistance to an RNA silencing agent; and
(ii) a second DNA editing agent for specifically editing the genome at the target sequence of interest; and
(c) culturing the cells under conditions that enrich for cells that comprise said selection marker, thereby selecting a cell which harbors the genome-editing event.

24-30. (canceled)

31. The method of claim 23, wherein said RNA silencing agent is siRNA.

32-40. (canceled)
